(19) United States
(12) Patent Application Publication
(10) Pub. No.: US 2012/0258976 A1
(43) Pub. Date: Oct. 11, 2012

(54) CRYSTALLINE PYRROLO[2,3-D]PYRIMIDINE COMPOUNDS

(75) Inventors: Brendan J. Murphy, Quaker Hill, CT (US); Timothy D. White, Zionsville, IN (US); Brian P. Chekal, Niantic, CT (US); Phillip J. Johnson, North Stonington, CT (US); Christopher James Foti, Waterford, CT (US); Leonid A. Margulis, West Hartford, CT (US)

(73) Assignee: Pfizer Inc., Madison, NJ (US)

(21) Appl. No.: 13/439,154

(22) Filed: Apr. 4, 2012

Related U.S. Application Data
(60) Provisional application No. 61/473,183, filed on Apr. 8, 2011.

(51) Int. Cl. A61K 31/519; A61P 17/06; A61P 17/00; C07D 487/04

(52) U.S. Cl. 514/265.1; 544/280

(57) ABSTRACT

The present invention discloses novel crystalline and non-crystalline forms of 3-((3R,4R)-4-methyl-3-[methyl-(7H-pyrrolo[2,3-d]pyrimidin-4-yl)-amino]-pip-eridin-1-yl)-3-oxopropionitrile, pharmaceutical composition containing the same, preparations thereof and the uses thereof.

### CROSS-REFERENCE TO RELATED APPLICATIONS

This application claims the benefit under 35 U.S.C. §119(e) of U.S. Provisional Patent Application Ser. No. 61/473,183, filed Apr. 8, 2011, which is incorporated herein by reference in its entirety for all purposes.

### FIELD OF THE INVENTION

The present invention relates to a crystalline form or a non-crystalline form of 3-((3R,4R)-4-methyl-3-[methyl-(7H-pyrrolo[2,3-d]pyrimidin-4-yl)-amino]-piperidin-1-yl)-3-oxopropionitrile. The present invention also relates to pharmaceutical compositions comprising a crystalline or non-crystalline form, and to methods for preparing such forms. The invention further relates to the use of a crystalline or non-crystalline form in the topical treatment of various diseases.

### BACKGROUND OF THE INVENTION

3-((3R,4R)-4-Methyl-3-[methyl-(7H-pyrrolo[2,3-d]pyrimidin-4-yl)-amino]-piperidin-1-yl)-3-oxopropionitrile has the chemical formula C₁₆H₂₀N₆O and the following structural formula

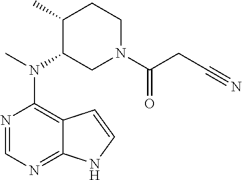

The synthesis of 3-((3R,4R)-4-methyl-3-[methyl-(7H-pyrrolo[2,3-d]-pyrimidin-4-yl)-amino]-piperidin-1-yl)-3-oxopropionitrile is described in WO 2001/42246 and WO 2002/096909, commonly assigned to the assignee of the present invention and which are incorporated herein by reference in their entirety. The preparation of 3-((3R,4R)-4-methyl-3-[methyl-(7H-pyrrolo[2,3-d]-pyrimidin-4-yl)-amino]-piperidin-1-yl)-3-oxopropionitrile mono citrate salt is described in U.S. Pat. No. 6,965,027. The crystalline or non-crystalline form of 3-((3R,4R)-4-methyl-3-[methyl-(7H-pyrrolo[2,3-d]pyrimidin-4-yl)-amino]-piperidin-1-yl)-3-oxopropionitrile free base, are also useful as inhibitors of protein kinases, such as the enzyme Janus Kinase (JAK) and as such are useful therapy as immunosuppressive agents for organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, Leukemia and other indications where immunosuppression would be desirable. The present invention relates to novel solid forms of the free base that demonstrate improved properties for use in a pharmaceutical dosage form, particularly for transdermal dosage forms.

Based on a chemical structure, one cannot predict with any degree of certainty whether a compound will crystallize, under what conditions it will crystallize, how many crystalline solid forms of the compound might exist, or the solid-state structure of any of those forms. A key characteristic of any crystalline drug is the polymorphic behavior of such a material. In general, crystalline forms of drugs are preferred over noncrystalline forms of drugs, in part, because of their superior stability. For example, in many situations, a noncrystalline drug converts to a crystalline drug form upon storage. Because noncrystalline and crystalline forms of a drug typically have differing physical properties and chemical properties, such interconversion may be undesirable for safety reasons in pharmaceutical usage. The different physical properties exhibited by different solid forms of a pharmaceutical compound can affect important pharmaceutical parameters such as storage, stability, compressibility, density (important in formulation and product manufacturing), and dissolution rates (important in determining bioavailability). Stability differences may result from changes in chemical reactivity (e.g., differential hydrolysis or oxidation, such that a dosage form comprising a certain polymorph can discolor more rapidly than a dosage form comprising a different polymorph), mechanical changes (e.g., tablets can crumble on storage as a kinetically favored crystalline form converts to thermodynamically more stable crystalline form), or both (e.g., tablets of one polymorph can be more susceptible to breakdown at high humidity). Solubility differences between polymorphs may, in extreme situations, result in transitions to crystalline forms that lack potency and/or that are toxic. In addition, the physical properties of a crystalline form may also be important in pharmaceutical processing. For example, a particular crystalline form may form solvates more readily or may be more difficult to filter and wash free of impurities than other crystalline forms (i.e., particle shape and size distribution might be different between one crystalline form relative to other forms).

There is no one ideal physical form of a drug because different physical forms provide different advantages. The search for the most stable form and for such other forms is arduous and the outcome is unpredictable. Thus it is important to seek a variety of unique drug forms, e.g. salts, polymorphs, non-crystalline forms, which may be used in various formulations. The selection of a drug form for a specific formulation or therapeutic application requires consideration of a variety of properties, and the best form for a particular application may be one which has one specific important good property while other properties may be acceptable or marginally acceptable.

The successful development of a drug requires that it meet certain general requirements to be a therapeutically effective treatment for patients. These requirements fall into two categories: (1) requirements for successful manufacture of dosage forms, and (2) requirements for successful drug delivery and disposition after the drug formulation has been administered to the patient.

Different crystalline solid forms of the same compound often possess different solid-state properties such as melting point, solubility, dissolution rate, hygroscopicity, powder flow, mechanical properties, chemical stability and physical stability. These solid-state properties may offer advantages in filtration, drying, and dosage form manufacturing unit operations. Thus, once different crystalline solid forms of the same compound have been identified, the optimum crystalline solid form under any given set of processing and manufacturing conditions may be determined as well as the different solid-state properties of each crystalline solid form.

Polymorphs of a molecule can be obtained by a number of methods known in the art. Such methods include, but are not limited to, melt recrystallization, melt cooling, solvent recrystallization, desolvation, rapid evaporation, rapid cooling, slow cooling, vapor diffusion and sublimation. Polymorphs can be detected, identified, classified and characterized using well-known techniques such as, but not limited to, differential scanning calorimetry (DSC), thermogravimetry (TGA), X-ray powder diffractometry (XRPD), single crystal X-ray diffractometry, solid state nuclear magnetic resonance (NMR), infrared (IR) spectroscopy, Raman spectroscopy, and hot-stage optical microscopy.

The present invention is directed to a crystalline and a non-crystalline form of 3-((3R,4R)-4-methyl-3-[methyl-(7H-pyrrolo[2,3-d]pyrimidin-4-yl)-amino]-piper-idin-1-yl)-3-oxopropionitrile free base. The invention is also directed to compositions, including pharmaceutical compositions, containing crystalline or non-crystalline 3-((3R,4R)-4-methyl-3-[methyl-(7H-pyrrolo[2,3-d]pyrimidin-4-yl)-amino]-piperidin-1-yl)-3-oxopropionitrile free base. The invention is further directed to processes for preparing crystalline and non-crystalline solid forms of 3-((3R,4R)-4-methyl-3-[methyl-(7H-pyrrolo[2,3-d]pyrimidin-4-yl)-amino]-piperidin-1-yl)-3-oxopropionitrile free base.

Because drug formulations, showing, for example, enhanced bioavailability or stability are consistently sought, there is an ongoing need for new or purer polymorphic forms of drug molecules. The polymorph of 3-((3R,4R)-4-methyl-3-[methyl-(7H-pyrrolo[2,3-d]pyrimidin-4-yl)-amino]-piperidin-1-yl)-3-oxopropionitrile described herein helps meet these and other needs.

### SUMMARY OF THE INVENTION

The present invention provides a crystalline form of 3-((3R,4R)-4-methyl-3-[methyl-(7H-pyrrolo[2,3-d]pyrimidin-4-yl)-amino]-piperidin-1-yl)-3-oxopropio-nitrile characterized by a powder X-ray diffraction pattern, solid state ¹³C nuclear magnetic resonance spectra, Raman spectra and FT-IR spectra.

The present invention provides a crystalline form, crystallized from a solvent system that includes 2-propanol, 2-propanol and tetrahydrofuran, tetrahydrofuran, ethanol and n-butanol, ethanol, n-butanol, 2-propanol and N,N-dimethylformamide, and tetrahydrofuran.

The present invention further provides a non-crystalline form of 3-((3R,4R)-4-methyl-3-[methyl-(7H-pyrrolo[2,3-d]pyrimidin-4-yl)-amino]-piperidin-1-yl)-3-oxopropionitrile characterized by a powder X-ray diffraction pattern, solid state ¹³C nuclear magnetic resonance spectrum, Raman spectrum and FT-IR spectrum.

The present invention also provides a pharmaceutical composition comprising 3-((3R,4R)-4-methyl-3-[methyl-(7H-pyrrolo[2,3-d]pyrimidin-4-yl)-amino]-piperidin-1-yl)-3-oxopropionitrile; one or more penetration enhancers; and a pharmaceutically acceptable carrier.

The present invention also provides a pharmaceutical composition comprising 3-((3R,4R)-4-methyl-3-[methyl-(7H-pyrrolo[2,3-d]pyrimidin-4-yl)-amino]-piperidin-1-yl)-3-oxopropionitrile, selected from the group consisting of a crystalline form or non-crystalline form; one or more penetration enhancers; and a pharmaceutically acceptable carrier.

The present invention also provides a method of treating a disease in a mammal, comprising administering to a mammal in need thereof a therapeutically effective amount of 3-((3R,4R)-4-methyl-3-[methyl-(7H-pyrrolo[2,3-d]pyrimidin-4-yl)-amino]-piperidin-1-yl)-3-oxopropionitrile, selected from the group consisting of a crystalline form or non-crystalline form or a pharmaceutically acceptable salt thereof or a pharmaceutical composition.

### BRIEF DESCRIPTION OF THE DRAWINGS

FIG. 1 depicts a calculated powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile at 23° C., containing approximately one equivalent of water.

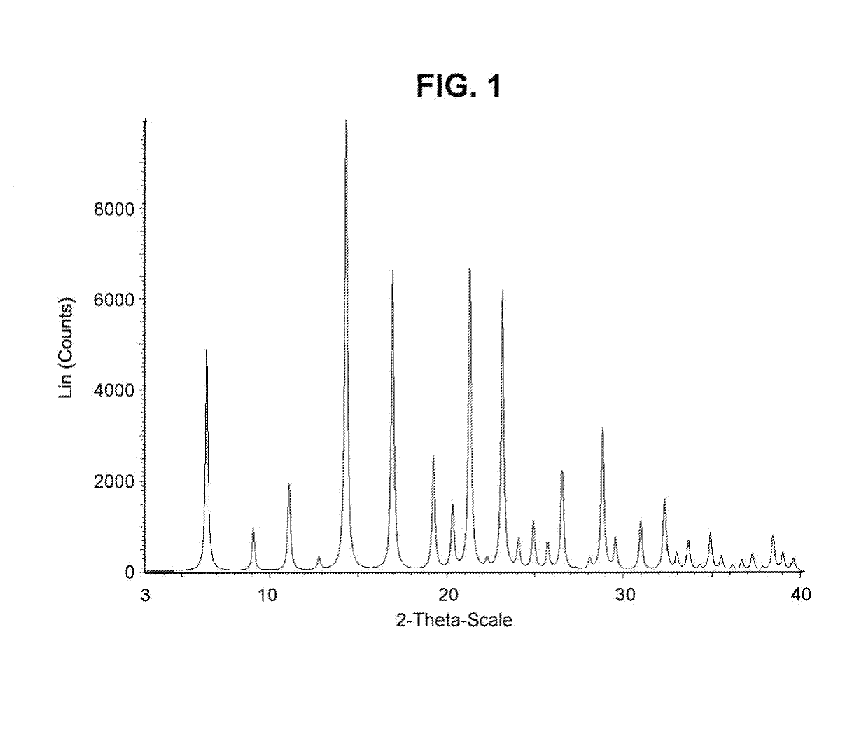

FIG. 2 depicts a calculated powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile at 120° C.

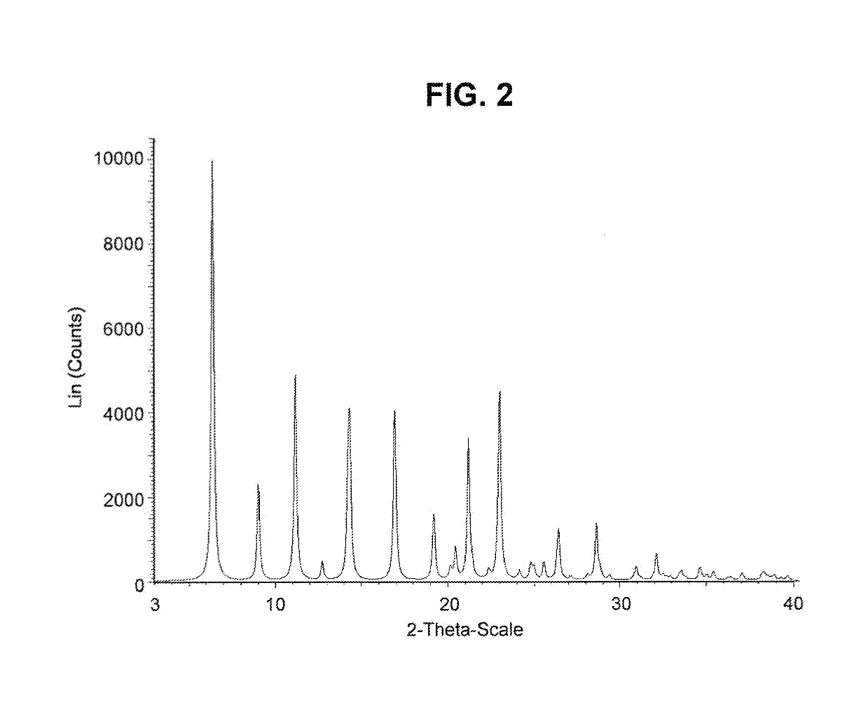

FIG. 3 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile prepared using process 1.

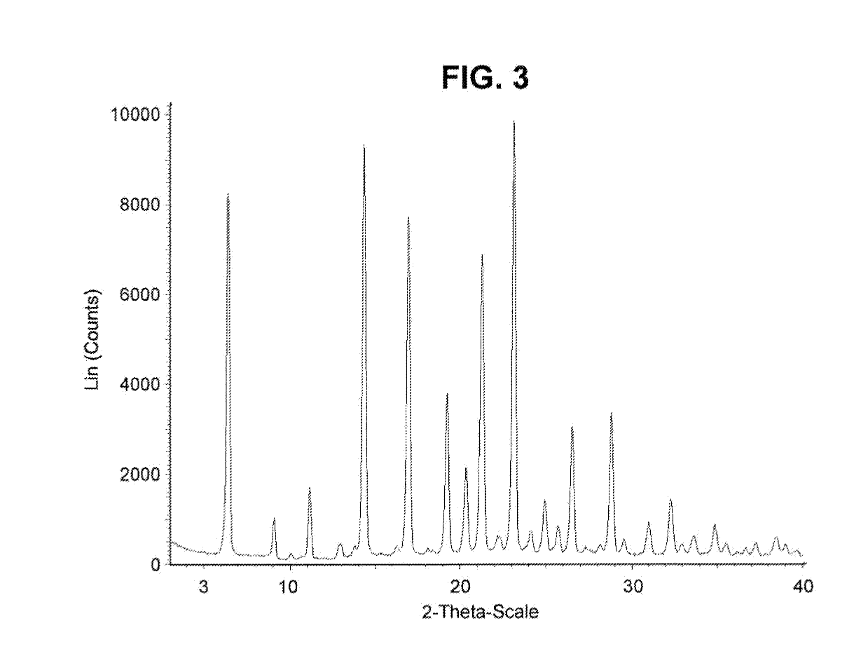

FIG. 4 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile prepared using process 2.

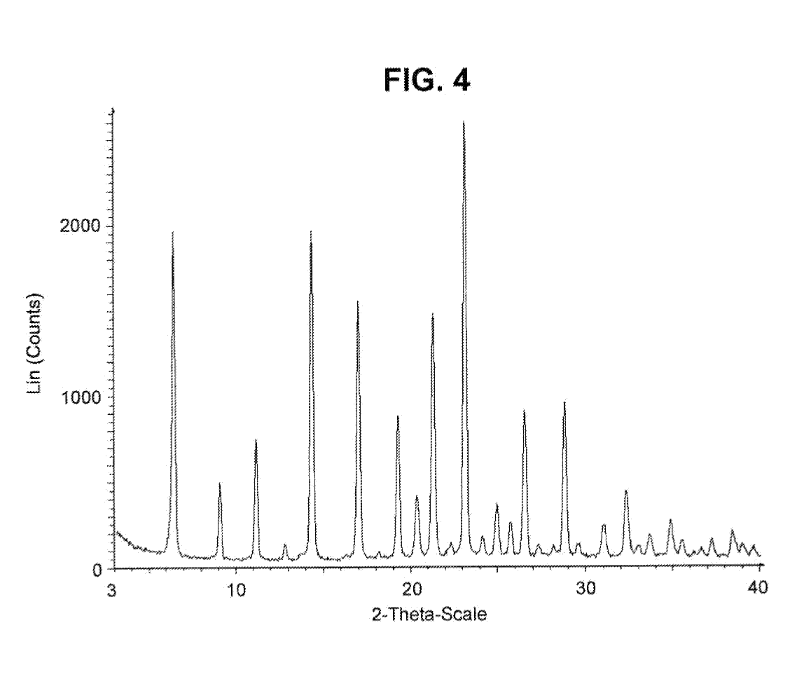

FIG. 5 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile prepared using process 3.

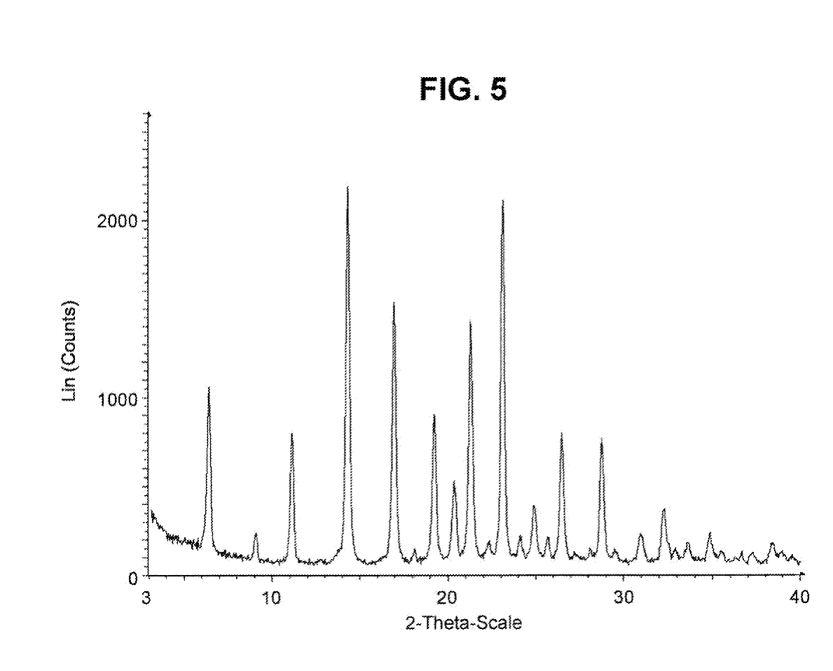

FIG. 6 depicts a Raman spectrum of the crystalline form of 3-((3R,4R)-4-methyl-3-[methyl-(7H-pyrrolo[2,3-d]pyrimidin-4-yl)-amino]-piperidin-1-yl)-3-oxopropionitrile prepared using process 2.

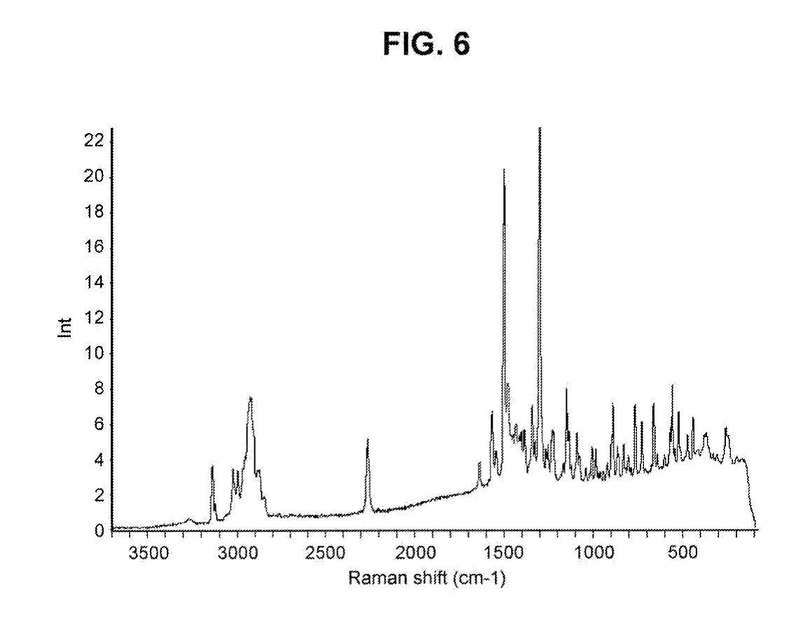

FIG. 7 depicts a FT-IR spectrum of the crystalline form of 3-((3R,4R)-4-methyl-3-[methyl-(7H-pyrrolo[2,3-d]pyrimidin-4-yl)-amino]-piperidin-1-yl)-3-oxopropionitrile prepared using process 2.

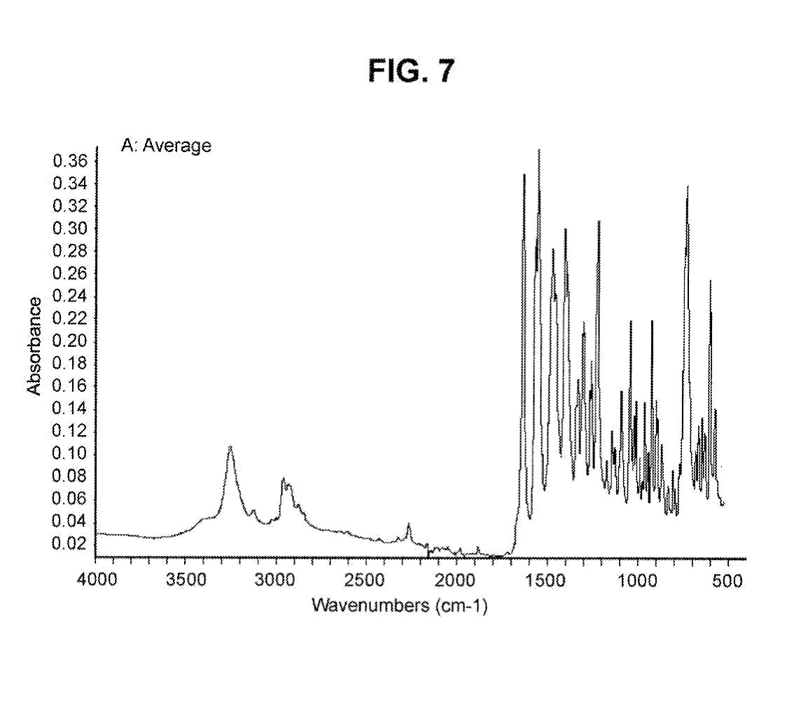

FIG. 8 depicts a solid state ¹³C nuclear magnetic resonance spectrum of the crystalline form of 3-((3R,4R)-4-methyl-3-[methyl-(7H-pyrrolo[2,3-d]-pyrimidin-4-yl)-amino]-piperidin-1-yl)-3-oxopropionitrile prepared using process 2. Spinning sidebands are noted with an asterisk.

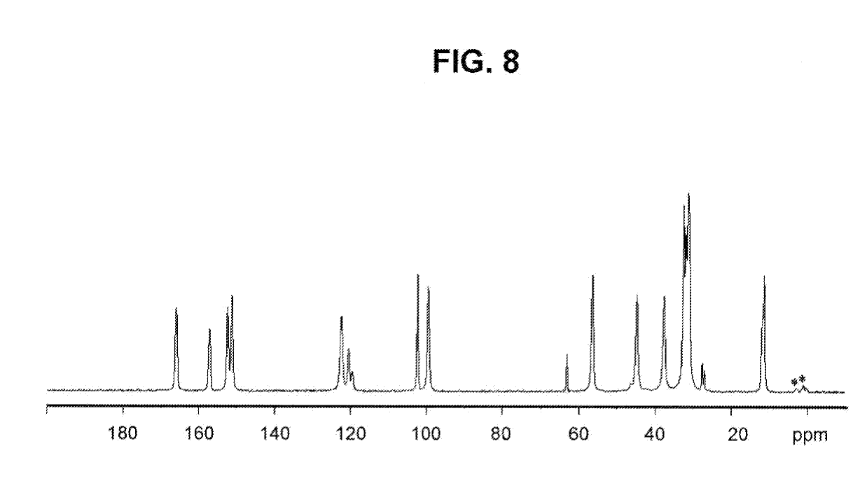

FIG. 9 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile containing methanol solvent.

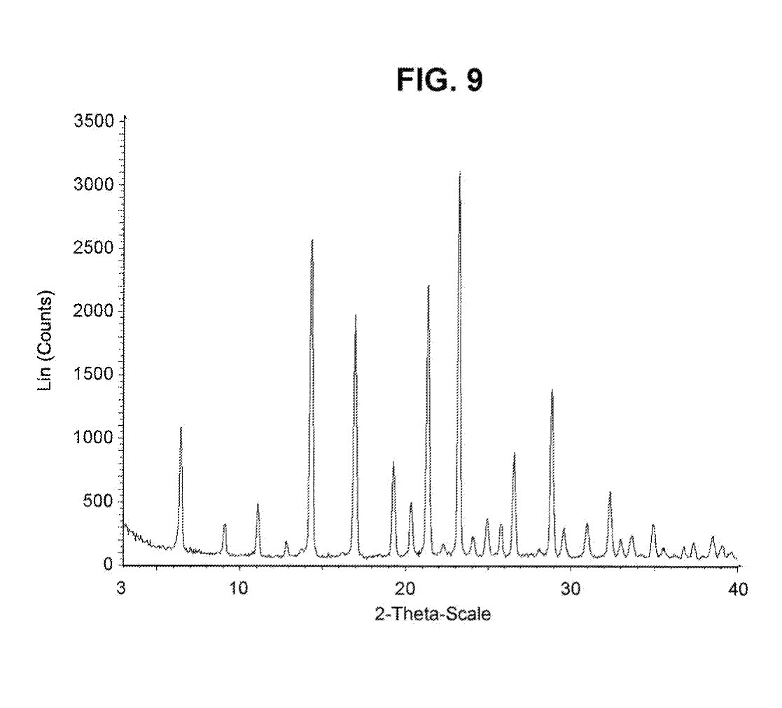

FIG. 10 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile containing acetone solvent.

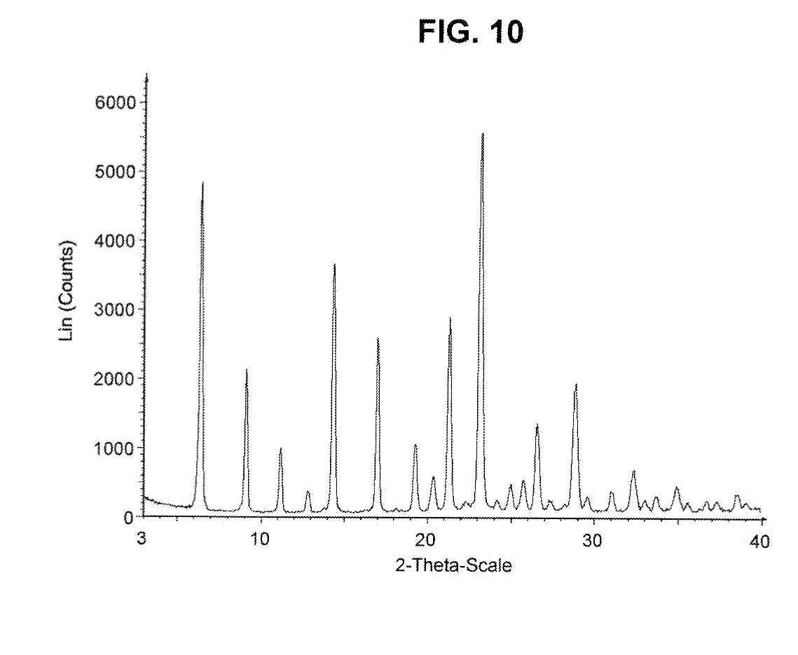

FIG. 11 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile containing 1-butanol and ethanol solvents.

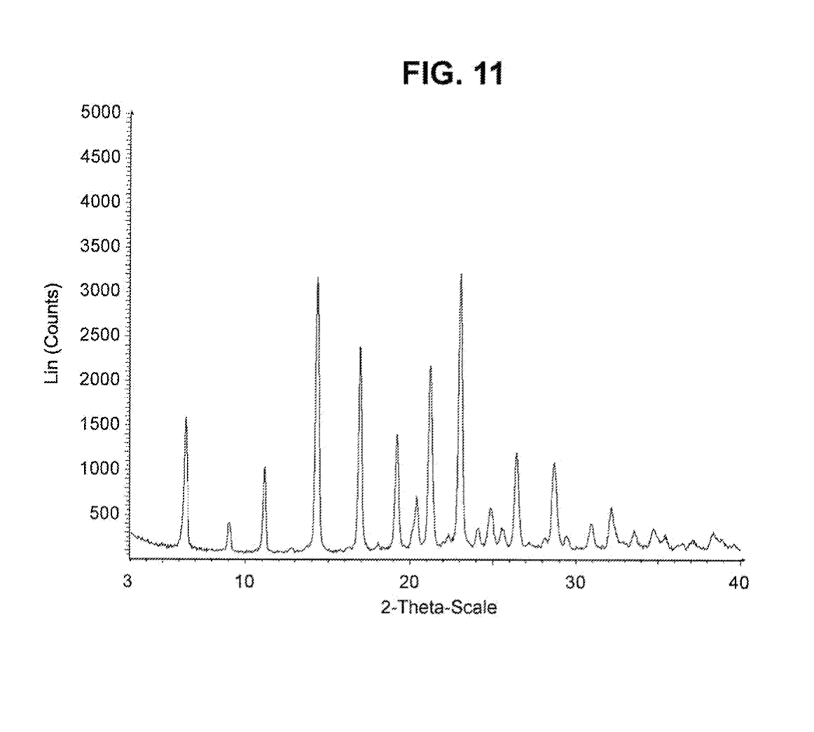

FIG. 12 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile containing N,N-dimethylformamide solvent.

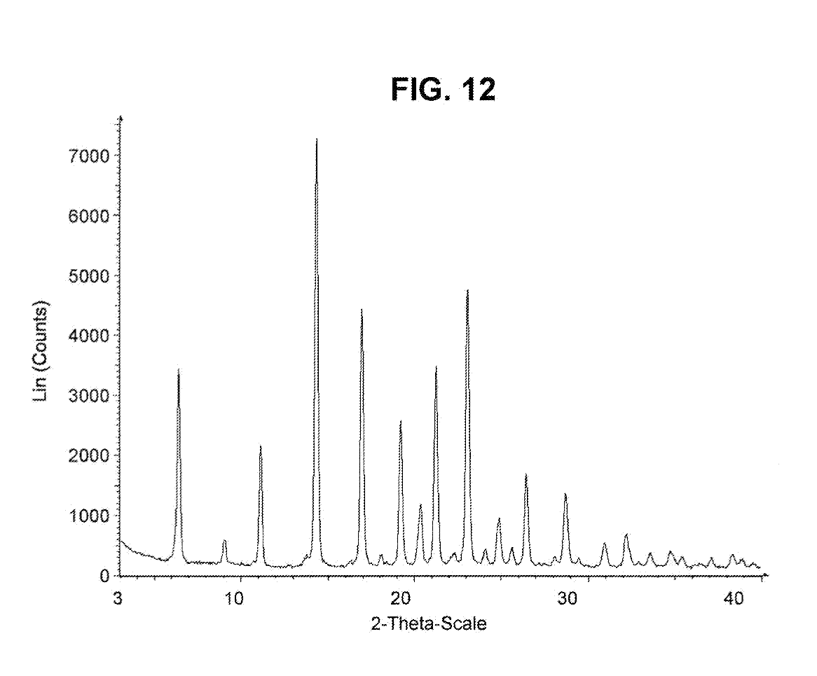

FIG. 13 depicts a powder X-ray diffraction pattern of the crystalline form of 3-((3R,4R)-4-methyl-3-[methyl-(7H-pyrrolo[2,3-d]pyrimidin-4-yl)-amino]-piperidin-1-yl)-3-oxopropionitrile containing tetrahydrofuran solvent.

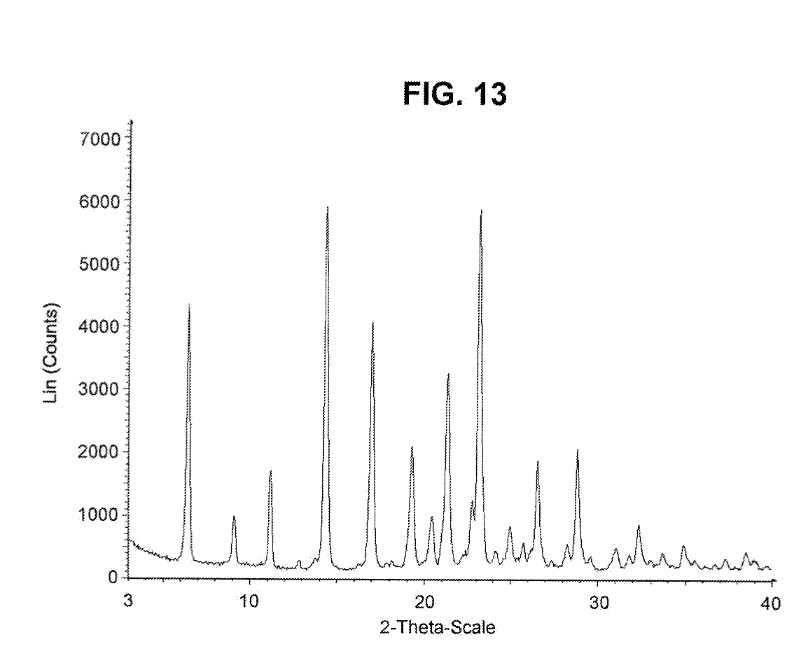

FIG. 14 depicts a solid state ¹³C nuclear magnetic resonance spectrum of the crystalline form of 3-((3R,4R)-4-methyl-3-[methyl-(7H-pyrrolo[2,3-d]-pyrimidin-4-yl)-amino]-piperidin-1-yl)-3-oxopropionitrile containing acetone solvent. Spinning sidebands are noted with an asterisk.

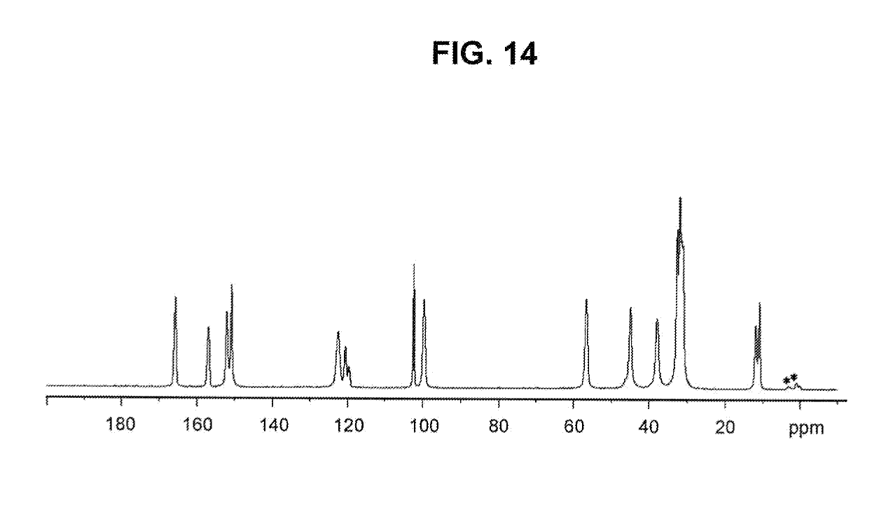

FIG. 15 depicts a solid state ¹³C nuclear magnetic resonance spectrum of the crystalline form of 3-((3R,4R)-4-methyl-3-[methyl-(7H-pyrrolo[2,3-d]-pyrimidin-4-yl)-amino]-piperidin-1-yl)-3-oxopropionitrile containing 1-butanol and ethanol solvents. Spinning sidebands are noted with an asterisk.

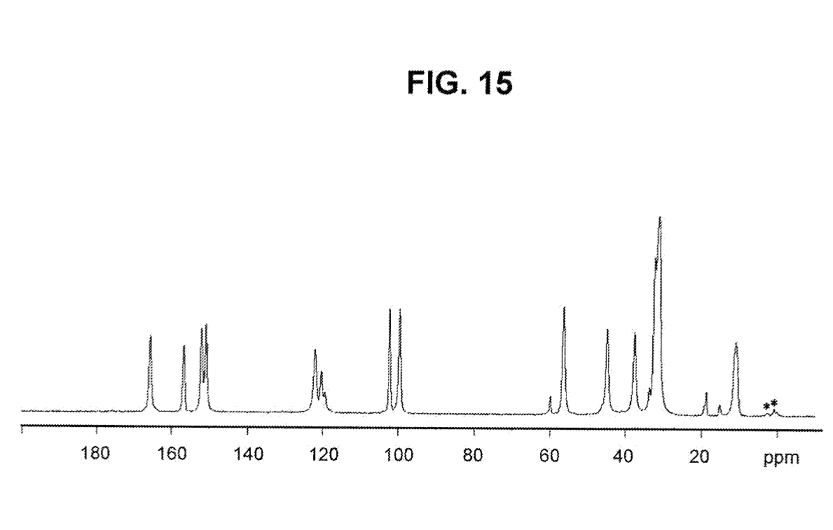

FIG. 16 depicts a solid state ¹³C nuclear magnetic resonance spectrum of the crystalline form of 3-((3R,4R)-4-methyl-3-[methyl-(7H-pyrrolo[2,3-d]-pyrimidin-4-yl)-amino]-piperidin-1-yl)-3-oxopropionitrile containing N,N-dimethylformamide solvent (Lot 121002-39-6). Spinning sidebands are noted with an asterisk.

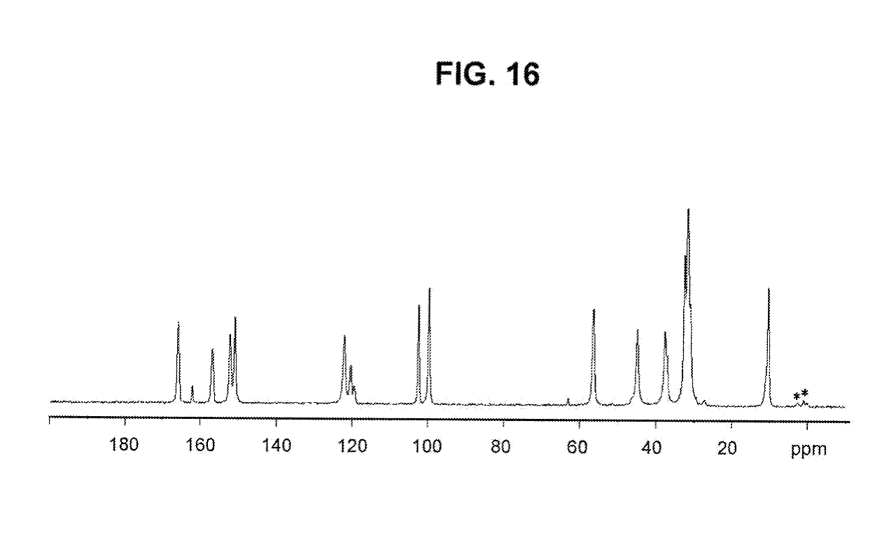

FIG. 17 depicts a solid state ¹³C nuclear magnetic resonance spectrum of the crystalline form of 3-((3R,4R)-4-methyl-3-[methyl-(7H-pyrrolo[2,3-d]-pyrimidin-4-yl)-amino]-piperidin-1-yl)-3-oxopropionitrile containing tetrahydrofuran solvent.

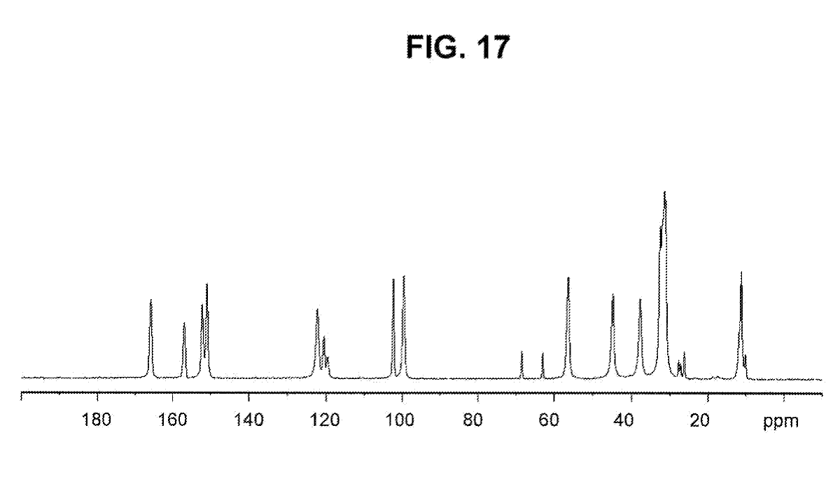

FIG. 18 depicts a powder X-ray diffraction pattern of the non-crystalline form of 3-((3R,4R)-4-methyl-3-[methyl-(7H-pyrrolo[2,3-d]pyrimidin-4-yl)-amino]-piperidin-1-yl)-3-oxopropionitrile.

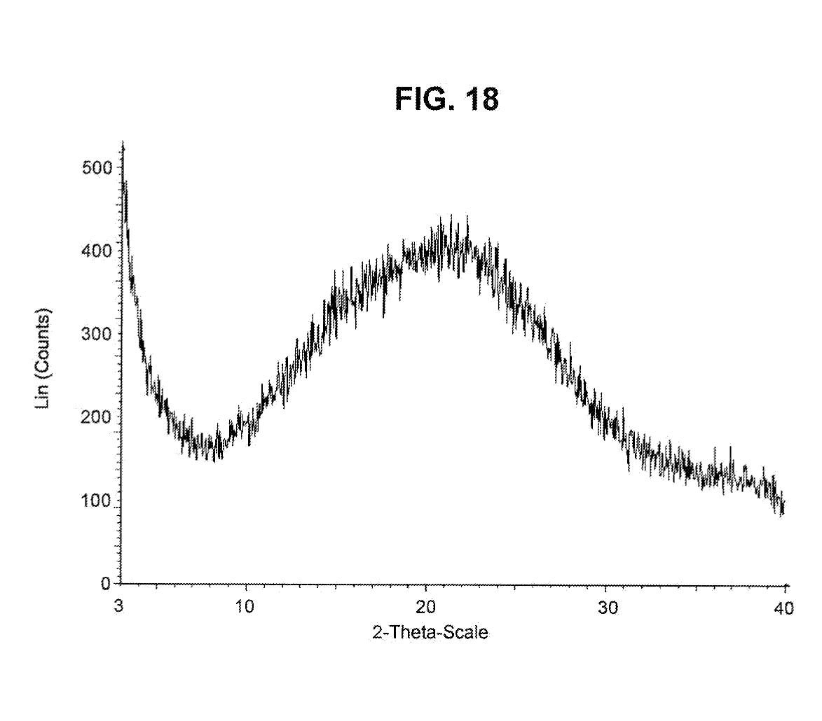

FIG. 19 depicts a solid state ¹³C nuclear magnetic resonance spectrum of the non-crystalline form of 3-((3R,4R)-4-methyl-3-[methyl-(7H-pyrrolo[2,3-d]-pyrimidin-4-yl)-amino]-piperidin-1-yl)-3-oxopropionitrile. Spinning sidebands are noted with an asterisk.

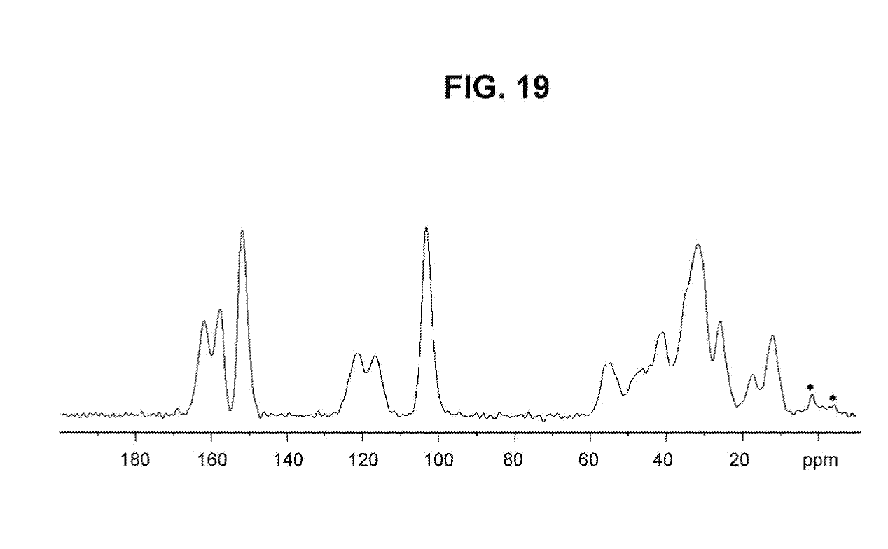

FIG. 20 depicts a Raman spectrum of the non-crystalline form of 3-((3R,4R)-4-methyl-3-[methyl-(7H-pyrrolo[2,3-d]pyrimidin-4-yl)-amino]-piperidin-1-yl)-3-oxopropionitrile.

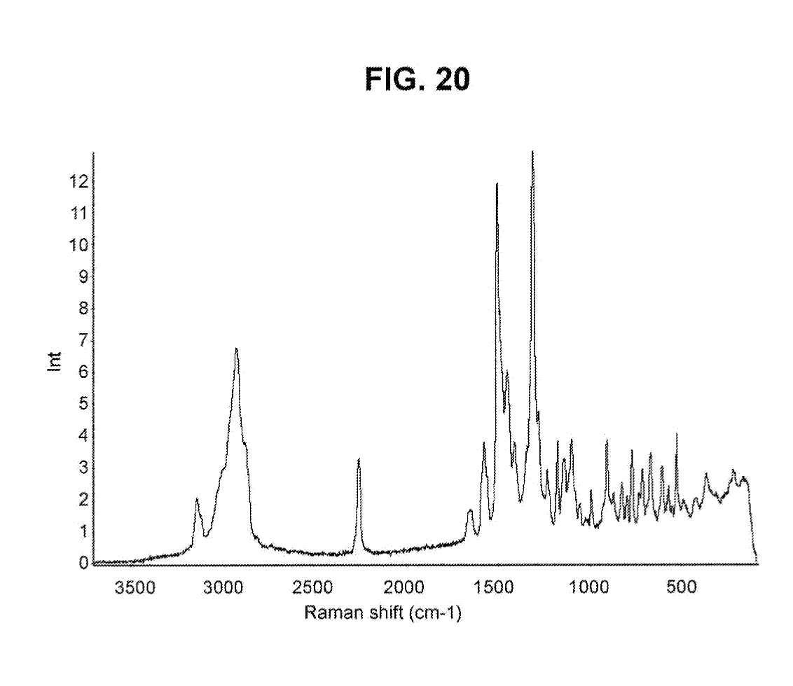

FIG. 21 depicts a FT-IR spectrum of the non-crystalline form of 3-((3R,4R)-4-methyl-3-[methyl-(7H-pyrrolo[2,3-d]pyrimidin-4-yl)-amino]-piperidin-1-yl)-3-oxopropionitrile.

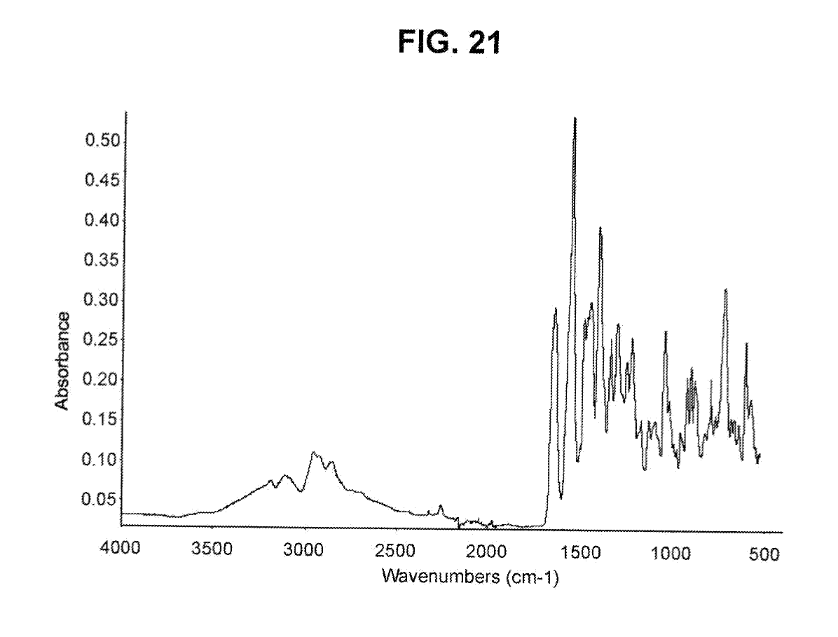

FIG. 22 depicts LSMean percent change from baseline (±SE) in TPSS by treatment group over time (FAS, No Imputation).

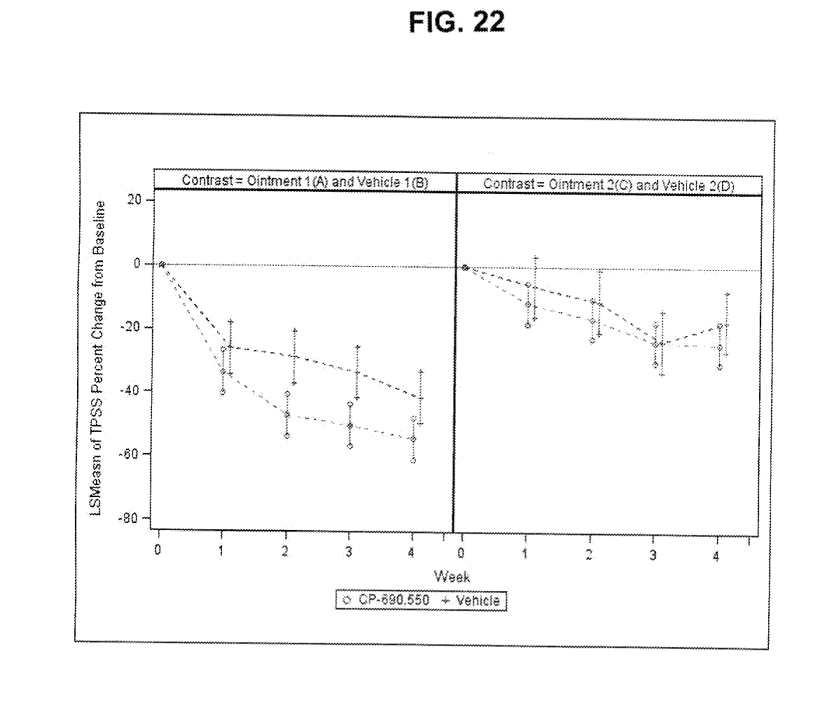

FIG. 23 depicts tofacitinib cumulative permeation through human cadaver skin for PEG-PEG ointments, μg/cm².

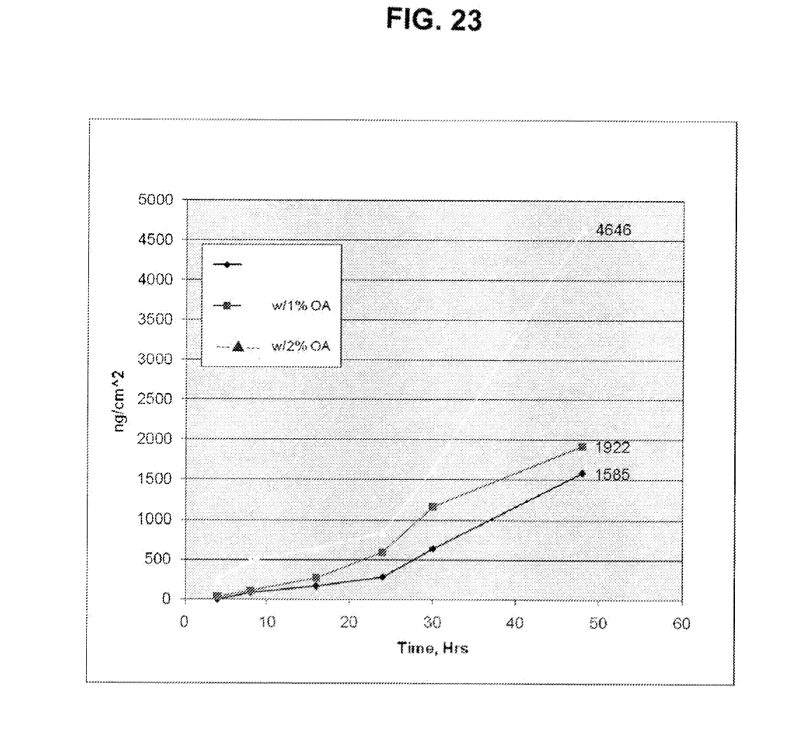

### DETAILED DESCRIPTION OF THE INVENTION

The present invention is directed to a crystalline form or a non-crystalline form of 3-((3R,4R)-4-methyl-3-[methyl-(7H-pyrrolo[2,3-d]pyrimidin-4-yl)amino]-piperidin-1-yl)-3-oxopropionitrile. The present invention is also directed to pharmaceutical compositions comprising the crystalline or non-crystalline forms, and to methods for preparing such forms. The invention is further directed to the use of the crystalline or non-crystalline forms in the treatment of various diseases.

There are a number of analytical methods one of ordinary skill in the art in solid-state chemistry can use to analyze solid forms. The term “analyze” as used herein means to obtain information about the solid-state structure of solid forms. For example, X-ray powder diffraction is a suitable technique for differentiating amorphous solid forms from crystalline solid forms and for characterizing and identifying crystalline solid forms of a compound. X-ray powder diffraction is also suitable for quantifying the amount of a crystalline solid form (or forms) in a mixture. In X-ray powder diffraction, X-rays are directed onto a crystal and the intensity of the diffracted X-rays is measured as a function of twice the angle between the X-ray source and the beam diffracted by the sample. The intensity of these diffracted X-rays can be plotted on a graph as peaks with the x-axis being twice the angle (this is known as the “2θ” angle) between the X-ray source and the diffracted X-rays and with the γ-axis being the intensity of the diffracted X-rays. This graph is called an X-ray powder diffraction pattern or powder pattern. Different crystalline solid forms exhibit different powder patterns because the location of the peaks on the x-axis is a property of the solid-state structure of the crystal.

Such powder patterns, or portions thereof, can be used as an identifying fingerprint for a crystalline solid form. Thus, one could take a powder pattern of an unknown sample and compare that powder pattern with a reference powder pattern. A positive match would mean that the unknown sample is of the same crystalline solid form as that of the reference. One could also analyze an unknown sample containing a mixture of solid forms by adding and subtracting powder patterns of known compounds.

When selecting peaks in a powder pattern to characterize a crystalline solid form or when using a reference powder pattern to identify a form, one identifies a peak or collection of peaks in one form that are not present in the other solid forms.

The term “characterize” as used herein means to select an appropriate set of data capable of distinguishing one solid form from another. That set of data in X-ray powder diffraction is the position of one or more peaks. Selecting which X-ray powder diffraction peaks define a particular form is said to characterize that form.

The term “identify” as used herein means taking a selection of characteristic data for a solid form and using those data to determine whether that form is present in a sample. In X-ray powder diffraction, those data are the x-axis positions of the one or more peaks characterizing the form in question as discussed above. For example, once one determines that a select number of X-ray diffraction peaks characterize a particular solid form, one can use those peaks to determine whether that form is present in a sample.

When characterizing and/or identifying crystalline solid forms of the same chemical compound with X-ray powder diffraction, it is often not necessary to use the entire powder pattern. A smaller subset of the entire powder pattern can often be used to perform the characterization and/or identification. By selecting a collection of peaks that differentiate the crystalline solid form from other crystalline solid forms of the compound, one can rely on those peaks to both characterize the form and to identify the form in, for example, an unknown mixture. Additional data can be added, such as from another analytical technique or additional peaks from the powder pattern, to characterize and/or identify the form should, for instance, additional polymorphs be identified later.

Due to differences in instruments, samples, and sample preparation, peak values is sometimes reported with the modifier “about” in front of the peak values. This is common practice in the solid-state chemical arts because of the variation inherent in peak values. A typical precision of the 2θ x-axis value of a peak in a powder pattern is on the order of plus or minus 0.2° 2θ. Thus, a powder diffraction peak that appears at “about 9.2° 2θ,” means that the peak could be between 9.0° 2θ and 9.4° 2θ when measured on most X-ray diffractometers under most conditions. Variability in peak intensity is a result of how individual crystals are oriented in the sample container with respect to the external X-ray source (known as “preferred orientation”). This orientation effect does not provide structural information about the crystal. X-ray powder diffraction is just one of several analytical techniques one may use to characterize and/or identify crystalline solid forms. Spectroscopic techniques such as Raman (including microscopic Raman), infrared, and solid state NMR spectroscopies may be used to characterize and/or identify crystalline solid forms. These techniques may also be used to quantify the amount of one or more crystalline solid forms in a mixture and peak values can also be reported with the modifier “about” in front of the peak values. A typical variability for a peak value associated with an FT-Raman and FT-Infrared measurement is on the order of plus or minus 2 cm⁻¹. A typical variability for a peak value associated with a ¹³C chemical shift is on the order of plus or minus 0.2 ppm for crystalline material. A typical variability for a value associated with a differential scanning calorimetry onset temperature is on the order of plus or minus 5° C.

The term “room temperature” as used herein refers to the temperature range of 20° C. to 23° C.

In the first aspect, the present invention comprises a crystalline form having one or more characteristics selected from the group consisting of:
I) an X-ray powder diffraction pattern containing the following 2θ values measured using Cu K_{α1} radiation (λ=1.54056 Å): 6.4, 14.3, and 17.0° 2θ±0.2° 2θ.
II) an X-ray powder diffraction pattern containing the following 2θ values measured using Cu K_{α1} radiation (λ=1.54056 Å): 6.4, 9.1, and 11.1° 2θ±0.2° 2θ.
III) a Raman spectrum containing the following wavenumber (cm⁻¹) values: 1305, 1504, and 2267 cm⁻¹±2 cm⁻¹.
IV) an infrared spectrum containing the following wavenumber (cm⁻¹) values: 1406, 1554, and 1635 cm⁻¹±2 cm⁻¹.
V) a ¹³C solid state NMR spectrum containing the following resonance (ppm) values: 157.0, 151.0, 102.4, 44.8, 32.7 ppm±0.2 ppm.
VI) a ¹³C solid state NMR spectrum containing the following resonance (ppm) values: 157.0, 151.0, 102.4, 63.1, 44.8, 32.7 ppm±0.2 ppm.
VII) a ¹³C solid state NMR spectrum containing the following resonance (ppm) values: 156.9, 151.0, 102.4, 68.6, 63.1, 44.9, 32.6 ppm±0.2 ppm.
VIII) a ¹³C solid state NMR spectrum containing the following resonance (ppm) values: 156.9, 151.0, 102.4, 68.6, 44.9, 32.6 ppm±0.2 ppm.
IX) a ¹³C solid state NMR spectrum containing the following resonance (ppm) values: 156.9, 151.0, 102.4, 60.1, 44.9, 32.6, 18.8 ppm±0.2 ppm.
X) a ¹³C solid state NMR spectrum containing the following resonance (ppm) values: 156.9, 151.0, 102.4, 60.1, 44.9, 32.6 ppm±0.2 ppm.
XI) a ¹³C solid state NMR spectrum containing the following resonance (ppm) values: 156.9, 151.0, 102.4, 44.9, 32.6, 18.8 ppm±0.2 ppm.
XII) a ¹³C solid state NMR spectrum containing the following resonance (ppm) values: 162.1, 156.8, 150.9, 102.5, 63.1, 44.9, 32.6 ppm±0.2 ppm.
XIII) a ¹³C solid state NMR spectrum containing the following resonance (ppm) values: 162.1, 156.8, 150.9, 102.5, 44.9, 32.6 ppm±0.2 ppm
XIV) an X-ray powder diffraction pattern containing the following 2θ values measured using Cu K_{α1} radiation (λ=1.54056 Å): 6.4, 14.3, 17.0±0.2° 2θ and a ¹³C solid state NMR spectrum containing the resonance (ppm) values selected from the group consisting of: 63.1, 63.1 and 68.6, 68.6, 18.8 and 60.1, 18.8, 60.1, 63.1 and 162.1, and 162.1 ppm±0.2 ppm.
XV) an X-ray powder diffraction pattern containing the following 2θ values measured using Cu K_{α1} radiation (λ=1.54056 Å): 6.4, 9.1, and 11.1±0.2° 2θ and a ¹³C solid state NMR spectrum containing the resonance (ppm) values selected from the group consisting of: 63.1, 63.1 and 68.6, 68.6, 18.8 and 60.1, 18.8, 60.1, 63.1 and 162.1, and 162.1 ppm±0.2 ppm.
XVI) an X-ray powder diffraction pattern containing the following 2θ values measured using Cu K_{α1} radiation (λ=1.54056 Å): 6.4, 14.3, 17.0° 2θ±0.2° 2θ and a 2-propanol level between 0.0, 2.6, 2.9, and 4.7% weight.
XVII) an X-ray powder diffraction pattern containing the following 2θ values measured using Cu K_{α1} radiation (λ=1.54056 Å): 6.4, 9.1, 11.1° 2θ±0.2° 2θ and a 2-propanol level between 0.0, 2.6, 2.9, and 4.7% weight.
XVIII) an X-ray powder diffraction pattern containing the following 2θ values measured using Cu K_{α1} radiation (λ=1.54056 Å): 6.4, 14.3, 17.0° 2θ±0.2° 2θ and a water level between 0.5 and 4.0% weight.
XIX) an X-ray powder diffraction pattern containing the following 2θ values measured using Cu K_{α1} radiation (λ=1.54056 Å): 6.4, 9.1, 11.1° 2θ±0.2° 2θ and a water level between 0.5 and 4.0% weight.
XX) a ¹³C solid state NMR spectrum containing the following resonance (ppm) values: 157.0, 151.0, 102.4, 44.8, 32.7 ppm±0.2 ppm and a 2-propanol level between 0.0, 2.6, 2.9, and 4.7% weight.
XXI) a ¹³C solid state NMR spectrum containing the following resonance (ppm) values: 157.0, 151.0, 102.4, 44.8, 32.7 ppm±0.2 ppm and a water level between 0.5 and 4.0% weight.
XXII) A crystallographic unit cell with dimensions and angles consisting of about: a=19.6-19.7 Å, b=19.6-19.7 Å, c=8.7 Å, α=90.0°, βp=90.0°, and γ=90.0.

In a second aspect, the present invention comprises a non-crystalline form having one or more characteristics selected from the group consisting of:
I) a ¹³C solid state NMR spectrum containing the following resonance (ppm) values: 161.9, 152.0, 103.3, 31.8, 26.0 ppm±0.2 ppm
II) a Raman spectrum containing the following wavenumber (cm⁻¹) values: 1311, 1506, and 2258 cm⁻¹±2 cm⁻¹.
III) a FTIR spectrum containing the following wavenumber (cm⁻¹) values: 1407, 1554, and 1647 cm⁻¹±2 cm⁻¹.
IV) A glass transition temperature of 87° C.

### Instrument and Analysis Methods:

Single Crystal X-ray Analysis at 23° C.: A sample crystal was prepared by evaporation of 1,4-dioxane/water (1:1, by volume) solution, as described by process 9. A representative crystal was surveyed and a 0.87 Å data set (maximum sin 0/X=0.57) was collected on a Bruker APEX II/R diffractometer. Atomic scattering factors were taken from the International Tables for Crystallography (Vol. C, pp. 219, 500, Kluwer Academic Publishers, 1992). Single crystal X-ray data were collected at 23° C. All crystallographic calculations were facilitated by the SHELXTL system (Version 5.1, Bruker AXS, 1997). A trial structure was obtained by direct methods and refined routinely. A difference map revealed a water of crystallization. Hydrogen positions were calculated wherever possible. The methyl hydrogens were located by difference Fourier techniques and then idealized. The hydrogens on nitrogen and oxygen were located by difference Fourier techniques and allowed to refine. The hydrogen parameters were added to the structure factor calculations but were not refined. The shifts calculated in the final cycles of least squares refinement were all less than 0.1 of the corresponding standard deviations. The final R-index was 4.15%. A final difference Fourier revealed no missing or misplaced electron density.

Single Crystal X-ray Analysis at 120° C.: A sample crystal utilized for X-ray analysis at 23° C. was also utilized for single crystal x-ray analysis at 120° C. A representative crystal was surveyed and a 1 Å data set (maximum sin 0/X=0.5) was collected on a Bruker APEX II/R diffractometer. Atomic scattering factors were taken from the International Tables for Crystallography (Vol. C, pp. 219, 500, Kluwer Academic Publishers, 1992). Single crystal X-ray data were collected at 120° C. All crystallographic calculations were facilitated by the SHELXTL system (Version 5.1, Bruker AXS, 1997). A trial structure was obtained by direct methods and refined routinely. A difference map revealed no water of crystallization. Hydrogen positions were calculated wherever possible. The methyl hydrogens were located by difference Fourier techniques and then idealized. The hydrogen parameters were added to the structure factor calculations but were not refined. The shifts calculated in the final cycles of least squares refinement were all less than 0.1 of the corresponding standard deviations. The final R-index was 9.29%. A final difference Fourier revealed no missing or misplaced electron density.

Calculated Powder Patterns: Powder patterns were calculated from single crystal X-ray data using the SHELXTL package of programs, including XFOG (SHELXTL, Bruker AXS, XFOG, Version 5.100, 1997) and XPOW (SHELXTL, Bruker AXS, XPOW, Version 5.102, 1997-2000). The appropriate wavelength needed for overlay graphics was added using the XCH file exchange program (SHELXTL, Bruker AXS, XCH, Version 5.0.4, 1995-2001).

Powder X-Ray Diffraction: The X-ray powder diffraction patterns were generated with a Siemens D5000 diffractometer using copper radiation. The instrument was equipped with a line focus X-ray tube. The tube voltage and amperage were set to 38 kV and 38 mA, respectively. The divergence and scattering slits were set at 1 mm, and the receiving slit was set at 0.6 mm. Diffracted Cu K_{α1} radiation (λ=1.54056 Å) was detected using a Sol-X energy dispersive X-ray detector. A theta two theta continuous scan at 2.4° 2θ/min. (1 sec/0.04° 2θ step) from 3.0 to 40.0° 2θ was used. An alumina standard (NIST standard reference material 1976) was analyzed to check the instrument alignment. Data were collected and analyzed using BRUKER AXS DIFFRAC PLUS software Version 2.0. Samples were prepared for analysis by placing them in a quartz holder.

PXRD Reflection Assignments: Eva Application 9.0 software was used to visualize and evaluate PXRD spectra. Peak values were assigned at the maximum intensity of a given reflection. All reflections exhibiting a relative intensity of greater than 10% are included within the following tables.

Differential Scanning calorimetry: The glass transition temperature of the non-crystalline form was determined using a Mettler-Toledo 821e differential scanning calorimeter under a 60 mL/minute Nitrogen purge. A sample of the non-crystalline form was placed in a 40 μL Aluminum pan. The pan was crimped and vented with a pinhole. A thermal treatment cycle was applied consecutively four times whereby the sample was heated from −10° C. to 200° C. at 20° C./minute and then cooled from 200° C. to −10° C. at −30° C./minute. A final thermal step followed whereby the sample was heated from −10° C. to 200° C. at 20° C./minute. The glass transition temperature was measured from the final heating segment of the thermal treatment using Mettler-Toledo STARe software Version 8.10 and reported herein by the measured midpoint.

Thermogravimetric Analysis with IR detection: Thermogravimetric analysis was conducted using a high resolution modulated 2950 thermogravimetric analyzer (TA Instruments) with TA Instrument Control 1.1A software. Instrument calibration was performed with calcium oxalate monohydrate. Samples of approximately 10 mg were weighed into aluminum pans (40 μL). Samples were heated from 30° C. to 300° C. at a heating rate of 5° C./min under a dry nitrogen purge (sample purge: 80 mL/min., balance purge: 20 mL/min.). Infrared detection of the evolved gases was enabled using a Thermo Nicolet Nexus 670 FTIR module in combination with a Nicolet magna-IR auxiliary experiment module. The transfer line temperature was maintained at 225° C. and cell temperature maintained at 250° C. during each experiment.

Solid State ¹³C Nuclear Magnetic Resonance Spectroscopy: A non-crystalline sample was prepared for analysis by packing it in a 4 mm ZrO₂ rotor. The proton decoupled ¹³C CPMAS (cross-polarization magic angle spinning experiment) spectrum was collected at ambient conditions on a Bruker-Biospin BL HFX CPMAS probe positioned into a wide-bore Bruker-Biospin Avance DSX 500 MHz NMR spectrometer. The rotor was oriented at the magic angle and spun at 15.0 kHz. The fast spinning speed minimized the intensity of the spinning side bands. The cross-polarization contact time was set to 2.0 ms. A proton decoupling field of approximately 91 kHz was applied. 632 scans were collected with recycle delay of 3.5 sec. The carbon spectrum was referenced using an external standard of crystalline adamantane, setting its upfield resonance to 29.5 ppm.

2-Propanolate (Process 2): The crystalline form was prepared for analysis by packing it in a 4 mm ZrO₂ rotor. The proton decoupled ¹³C CPMAS (cross-polarization magic angle spinning experiment) spectrum was collected at ambient conditions on a Bruker-Biospin 4 mm HFX CPMAS probe positioned into a wide-bore Bruker-Biospin Avance DSX 500 MHz NMR spectrometer. The rotor was oriented at the magic angle and spun at 15.0 kHz. The fast spinning speed minimized the intensity of the spinning side bands. The cross-polarization contact time was set to 2.0 ms. A proton decoupling field of approximately 87 kHz was applied. 2,468 scans were collected with recycle delay of 1.3 sec. The carbon spectrum was referenced using an external standard of crystalline adamantane, setting its upfield resonance to 29.5 ppm.

Acetone Solvate (Process 5): The crystalline form was prepared for analysis by packing it in a 4 mm ZrO₂ rotor. The proton decoupled ¹³C CPMAS (cross-polarization magic angle spinning experiment) spectrum was collected at ambient conditions on a Bruker-Biospin 4 mm HFX CPMAS probe positioned into a wide-bore Bruker-Biospin Avance DSX 500 MHz NMR spectrometer. The rotor was oriented at the magic angle and spun at 15.0 kHz. The fast spinning speed minimized the intensity of the spinning side bands. The cross-polarization contact time was set to 2.0 ms. A proton decoupling field of approximately 86 kHz was applied. 11,332 scans were collected with recycle delay of 1.8 sec. The carbon spectrum was referenced using an external standard of crystalline adamantane, setting its upfield resonance to 29.5 ppm.

n-Butanolate/ethanolate (Process 6): The crystalline form was prepared for analysis by packing it in a 4 mm ZrO₂ rotor. The proton decoupled ¹³C CPMAS (cross-polarization magic angle spinning experiment) spectrum was collected at ambient conditions on a Bruker-Biospin 4 mm HFX CPMAS probe positioned into a wide-bore Bruker-Biospin Avance DSX 500 MHz NMR spectrometer. The rotor was oriented at the magic angle and spun at 15.0 kHz. The fast spinning speed minimized the intensity of the spinning side bands. The cross-polarization contact time was set to 2.0 ms. A proton decoupling field of approximately 86 kHz was applied. 8,000 scans were collected with recycle delay of 5.5 seconds. The carbon spectrum was referenced using an external standard of crystalline adamantane, setting its upfield resonance to 29.5 ppm.

Dimethylformamide Solvate (Process 7): The crystalline form was prepared for analysis by packing it in a 4 mm ZrO₂ rotor. The proton decoupled ¹³C CPMAS (cross-polarization magic angle spinning experiment) spectrum was collected at ambient conditions on a Bruker-Biospin 4 mm HFX CPMAS probe positioned into a wide-bore Bruker-Biospin Avance DSX 500 MHz NMR spectrometer. The rotor was oriented at the magic angle and spun at 15.0 kHz. The fast spinning speed minimized the intensity of the spinning side bands. The cross-polarization contact time was set to 2.0 ms. A proton decoupling field of approximately 87 kHz was applied. 1,144 scans were collected with recycle delay of 10 sec. The carbon spectrum was referenced using an external standard of crystalline adamantane, setting its upfield resonance to 29.5 ppm.

Tetrahydrofuran Solvate (Process 8): The crystalline form was prepared for analysis by packing it in a 4 mm ZrO₂ rotor. The proton decoupled ¹³C CPMAS (cross-polarization magic angle spinning experiment) spectrum was collected at ambient conditions on a Bruker-Biospin 4 mm HFX CPMAS probe positioned into wide-bore Bruker-Biospin Avance DSX 500 MHz NMR spectrometer. The rotor was oriented at the magic angle and spun at 15.0 kHz. The fast spinning speed minimized the intensity of the spinning side bands. The cross-polarization contact time was set to 2.0 ms. A proton decoupling field of approximately 87 kHz was applied. 5,120 scans were collected with recycle delay of 5.0 sec. The carbon spectrum was referenced using an external standard of crystalline adamantane, setting its upfield resonance to 29.5 ppm.

Infrared Spectroscopy: IR spectra were acquired using a ThermoNicolet Magna 560 FTIR spectrometer equipped with a KBr beamsplitter and a d-TGS KBr detector. A Specac Golden Gate Mk II single reflection diamond ATR accessory was used for sampling. A nitrogen purge was connected to the IR bench as well as the ATR accessory. Prior to data acquisition, instrument performance and calibration verifications were conducted using polystyrene. An air background was collected prior to each sample by collecting spectra with the Golden Gate ATR anvil in the raised position. Powder samples were compressed against the diamond window by the Golden Gate anvil using a torque wrench to apply 20 cN·m of torque to the anvil compression control knob. The ATR accessory was cleaned prior to scanning of each new sample. Spectra were collected at 2 cm⁻¹ resolution using 128 co-added scans and a collection range of 4000-525 cm⁻¹. Happ-Genzel apodization was used. Three separate sample spectra were collected, with decompression and mixing of the powder conducted after each spectral collection. The separate spectra for each sample were averaged together. Band positions were assigned manually at peak maximum values. With this method, the positional accuracy of these peaks is +/−2 cm⁻¹. It should be noted that diamond spectral features in the region between 2400-1900 cm⁻¹ are present in all spectra run by the Golden Gate d-ATR (Ferrer, N.; Nogués-Carulla, J. M. Diamond and Related Materials 1996, 5, 598-602. Thongnopkun, P.; Ekgasit, S. Diamond and Related Materials 2005, 14, 1592-1599. Pike Technologies Technical Note: Pike Reflections, Winter 2002, Vol. 7/1; www.piketech.com).

Raman Spectroscopy: Raman spectra were collected using a ThermoNicolet 960 FT-Raman spectrometer equipped with a 1064 nm NdYAG laser and InGaAs detector. A data collection range of 4000-100 cm⁻¹ was used. All spectra were recorded using 2 cm⁻¹ resolution, Happ-Genzel apodization, and 100 co-added scans. Prior to data acquisition, instrument performance and calibration verifications were conducted using polystyrene. Samples were analyzed in glass NMR tubes. Three separate spectra were recorded for each sample, with 45° sample rotation between spectral collections. The displayed spectra result from the arithmetic mean of the three individual spectra. Band positions were assigned manually at peak maximum values. With this method, the positional accuracy of these peaks is +/−2 cm⁻¹. The crystalline form spectra were collected using 0.5 W of laser power and the non-crystalline form spectra were collected using 1.0 W of laser power.

Karl Fischer Analysis: Water content values were measured using a Binkmann's model 737 Karl Fischer Coulometer equipped with a Sartorius BP221S balance.

Residual Solvent Analysis: Solvent content values were measured using a gas chromatograph equipped with a flame ionization detector and split injection capability for column operation, and an automated headspace sampler. Each sample was prepared for analysis by accurately weighing 40 mg of solid into a headspace vial. 4.0 mL of N,N-dimethylacetamide was added to the vial and the vial immediately sealed with a septum and a crimp cap. A blank as well as the appropriate solvent standards were prepared and tested prior to evaluation of each sample.

The present invention provides a crystalline form or a non-crystalline form of 3-((3R,4R)-4-methyl-3-[methyl-(7H-pyrrolo[2,3-d]pyrimidin-4-yl)-amino]-pip-eridin-1-yl)-3-oxopropionitrile which can be identified by one or more solid state analytical methods.

PXRD peak list for the crystalline form containing approximately one equivalent of water at 23° C. is shown in Table 1.

TABLE 1

| Reflection | Relative |
|---|---|
| (°2θ) | Intensity (%) |
| 6.4 | 49 |
| 9.0 | 10 |
| 11.1 | 19 |
| 14.3 | 100 |
| 17.0 | 66 |
| 19.2 | 25 |
| 20.3 | 15 |
| 21.3 | 67 |
| 23.2 | 62 |
| 24.9 | 11 |
| 26.6 | 22 |
| 28.9 | 31 |
| 31.0 | 11 |
| 32.4 | 16 |

PXRD peak list for the crystalline form at 120° C. is shown in Table 2.

TABLE 2

| Reflection | Relative |
|---|---|
| (°2θ) | Intensity (%) |
| 6.3 | 100 |
| 9.0 | 23 |
| 11.1 | 49 |
| 14.3 | 41 |
| 16.9 | 40 |
| 19.2 | 16 |
| 21.2 | 34 |
| 23.0 | 45 |
| 26.4 | 13 |
| 28.6 | 14 |

PXRD peak list for the crystalline form, prepared using process 1 is shown in Table 3.

TABLE 3

| Reflection | Relative |
|---|---|
| (°2θ) | Intensity (%) |
| 6.4 | 84 |
| 9.1 | 10 |
| 11.1 | 17 |
| 14.3 | 95 |
| 17.0 | 79 |
| 19.3 | 39 |
| 20.3 | 22 |
| 21.3 | 70 |
| 23.1 | 100 |
| 24.9 | 14 |
| 26.5 | 31 |
| 28.8 | 34 |
| 32.3 | 15 |

PXRD peak list for the crystalline form, prepared using process 2 is shown in Table 4

TABLE 4

| Reflection | Relative |
|---|---|
| (°2θ) | Intensity (%) |
| 6.4 | 75 |
| 9.0 | 19 |
| 11.2 | 29 |
| 14.4 | 75 |
| 17.0 | 59 |
| 19.3 | 34 |
| 20.4 | 16 |
| 21.4 | 57 |
| 23.2 | 100 |
| 25.0 | 14 |
| 25.7 | 10 |
| 26.6 | 35 |
| 28.8 | 37 |
| 32.3 | 17 |
| 34.9 | 10 |

PXRD peak list for the crystalline form, prepared using process 3 is shown in Table 5.

TABLE 5

| Reflection | Relative |
|---|---|
| (°2θ) | Intensity (%) |
| 6.4 | 48 |
| 9.0 | 11 |
| 11.1 | 36 |
| 14.3 | 100 |
| 16.9 | 70 |
| 19.2 | 41 |
| 20.3 | 24 |
| 21.3 | 65 |
| 23.1 | 96 |
| 24.1 | 10 |
| 24.9 | 18 |
| 25.7 | 10 |
| 26.5 | 36 |
| 28.8 | 35 |
| 31.0 | 10 |
| 32.3 | 17 |
| 34.9 | 11 |

Raman peak list for the crystalline form, prepared using process 2 is shown in Table 6.

TABLE 6

| Wavenumber | Relative |
|---|---|
| (cm⁻¹) | Intensity |
| 201 | Weak |
| 251 | Weak |
| 263 | Weak |
| 313 | Weak |
| 334 | Weak |
| 360 | Weak |
| 371 | Weak |
| 382 | Weak |
| 397 | Weak |
| 416 | Weak |
| 420 | Weak |
| 446 | Weak |
| 478 | Weak |
| 493 | Weak |
| 517 | Weak |
| 526 | Weak |
| 548 | Weak |
| 562 | Medium |
| 575 | Weak |
| 606 | Weak |
| 646 | Weak |
| 666 | Weak |
| 680 | Weak |
| 714 | Weak |
| 716 | Weak |
| 733 | Weak |
| 770 | Weak |
| 784 | Weak |
| 790 | Weak |
| 796 | Weak |
| 808 | Weak |
| 821 | Weak |
| 825 | Weak |
| 834 | Weak |
| 844 | Weak |
| 848 | Weak |
| 863 | Weak |
| 870 | Weak |
| 895 | Weak |
| 902 | Weak |
| 913 | Weak |
| 925 | Weak |
| 937 | Weak |
| 946 | Weak |
| 955 | Weak |
| 966 | Weak |
| 976 | Weak |
| 990 | Weak |
| 1010 | Weak |
| 1023 | Weak |
| 1035 | Weak |
| 1046 | Weak |
| 1057 | Weak |
| 1066 | Weak |
| 1081 | Weak |
| 1095 | Weak |
| 1115 | Weak |
| 1129 | Weak |
| 1142 | Weak |
| 1152 | Medium |
| 1172 | Weak |
| 1184 | Weak |
| 1202 | Weak |
| 1229 | Weak |
| 1235 | Weak |
| 1259 | Weak |
| 1270 | Weak |
| 1286 | Weak |
| 1305 | Strong |
| 1330 | Weak |
| 1347 | Weak |
| 1391 | Weak |
| 1407 | Weak |
| 1419 | Weak |
| 1438 | Weak |
| 1441 | Weak |
| 1454 | Weak |
| 1466 | Weak |
| 1483 | Medium |
| 1504 | Strong |
| 1551 | Weak |
| 1572 | Weak |
| 1641 | Weak |
| 2267 | Weak |
| 2767 | Weak |
| 2804 | Weak |
| 2851 | Weak |
| 2880 | Weak |
| 2890 | Weak |
| 2926 | Weak |
| 2936 | Weak |
| 2963 | Weak |
| 2974 | Weak |
| 3002 | Weak |
| 3031 | Weak |
| 3068 | Weak |
| 3129 | Weak |
| 3144 | Weak |
| 3260 | Weak |
| 3276 | Weak |

FT-IR peak list for the crystalline form, prepared using process 2 is shown in Table 7.

TABLE 7

| Wavenumber | Relative |
|---|---|
| (cm⁻¹) | Intensity |
| 546 | Weak |
| 577 | Medium |
| 604 | Strong |
| 631 | Medium |
| 644 | Medium |
| 665 | Medium |
| 681 | Weak |
| 731 | Strong |
| 740 | Strong |
| 769 | Weak |
| 795 | Weak |
| 808 | Weak |
| 822 | Weak |
| 833 | Weak |
| 870 | Weak |
| 895 | Medium |
| 902 | Medium |
| 924 | Medium |
| 943 | Weak |
| 964 | Medium |
| 976 | Weak |
| 989 | Weak |
| 1011 | Medium |
| 1022 | Medium |
| 1045 | Medium |
| 1093 | Medium |
| 1128 | Weak |
| 1142 | Weak |
| 1172 | Weak |
| 1225 | Strong |
| 1259 | Medium |
| 1267 | Medium |
| 1304 | Medium |
| 1333 | Medium |
| 1342 | Medium |
| 1394 | Strong |
| 1406 | Strong |
| 1460 | Medium |
| 1475 | Strong |
| 1554 | Strong |
| 1568 | Strong |
| 1635 | Strong |
| 1722 | Weak |
| 1755 | Weak |
| 1774 | Weak |
| 1797 | Weak |
| 1823 | Weak |
| 1832 | Weak |
| 1852 | Weak |
| 1880 | Weak |
| 2430 | Weak |
| 2494 | Weak |
| 2606 | Weak |
| 2656 | Weak |
| 2855 | Weak |
| 2879 | Weak |
| 2926 | Weak |
| 2935 | Weak |
| 2963 | Weak |
| 2999 | Weak |
| 3021 | Weak |
| 3128 | Weak |
| 3255 | Weak |

ss ¹³C NMR peak list for the crystalline form, prepared using process 2 is shown in Table 8.

TABLE 8

| ¹³C Chemical | |
|---|---|
| Shifts [ppm]^{a} | Intensity^{b} |
| 165.9 | 5.00 |
| 157.0 | 3.65 |
| 152.2 | 4.95 |
| 151.0 | 5.65 |
| 122.4 | 4.42 |
| 120.5 | 2.46 |
| 119.5 | 1.07 |
| 102.4 | 7.09 |
| 99.6 | 6.23 |
| 63.1 | 2.14 |
| 56.4 | 6.98 |
| 44.8 | 5.79 |
| 37.7 | 5.67 |
| 32.7 | 11.21 |
| 32.2 | 9.44 |
| 31.6 | 12.00 |
| 31.3* | Peak shoulder |
| 27.7 | 1.62 |
| 27.2 | 1.17 |
| 12.0* | Peak shoulder |
| 11.7 | 4.47 |
| 11.3 | 7.07 |
| 10.8* | Peak shoulder |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.
*Peak shoulder

PXRD peak list for the crystalline form, containing methanol solvent is shown in Table 9.

TABLE 9

| Reflection | Relative |
|---|---|
| (°2θ) | Intensity (%) |
| 6.4 | 35 |
| 9.1 | 11 |
| 11.1 | 16 |
| 14.3 | 83 |
| 16.9 | 64 |
| 19.2 | 26 |
| 20.3 | 16 |
| 21.3 | 71 |
| 23.2 | 100 |
| 24.9 | 12 |
| 25.7 | 11 |
| 26.6 | 28 |
| 28.9 | 45 |
| 31.0 | 11 |
| 32.4 | 19 |
| 34.9 | 11 |

PXRD peak list for the crystalline form, containing acetone solvent is shown in Table 10.

TABLE 10

| Reflection | Relative |
|---|---|
| (°2θ) | Intensity (%) |
| 6.4 | 87 |
| 9.0 | 38 |
| 11.2 | 18 |
| 14.3 | 66 |
| 17.0 | 47 |
| 19.3 | 19 |
| 21.3 | 52 |
| 23.2 | 100 |
| 26.6 | 24 |
| 28.9 | 35 |

PXRD peak list for the crystalline form, containing 1-butanol and ethanol solvents is shown in Table 11.

TABLE 11

| Reflection | Relative |
|---|---|
| (°2θ) | Intensity (%) |
| 6.4 | 49 |
| 9.0 | 13 |
| 11.2 | 32 |
| 14.3 | 99 |
| 17.0 | 75 |
| 19.2 | 44 |
| 20.4 | 22 |
| 21.2 | 68 |
| 23.1 | 100 |
| 24.2 | 11 |
| 24.9 | 18 |
| 25.6 | 11 |
| 26.5 | 37 |
| 28.7 | 34 |
| 31.0 | 13 |
| 32.2 | 18 |
| 33.6 | 10 |
| 34.8 | 11 |
| 38.4 | 10 |

PXRD peak list for the crystalline form, N,N-dimethylformamide solvent is shown in Table 12.

TABLE 12

| Reflection | Relative |
|---|---|
| (°2θ) | Intensity (%) |
| 6.4 | 47 |
| 9.0 | 8 |
| 11.1 | 30 |
| 14.3 | 100 |
| 16.9 | 61 |
| 19.2 | 35 |
| 20.4 | 16 |
| 21.2 | 48 |
| 23.1 | 65 |
| 24.9 | 13 |
| 26.4 | 23 |
| 28.7 | 19 |

PXRD peak list for the crystalline form, containing tetrahydrofuran solvent is shown in Table 13.

TABLE 13

| Reflection | Relative |
|---|---|
| (°2θ) | Intensity (%) |
| 6.4 | 73 |
| 9.0 | 17 |
| 11.1 | 29 |
| 14.3 | 100 |
| 17.0 | 69 |
| 19.3 | 35 |
| 20.4 | 17 |
| 21.3 | 55 |
| 22.7 | 21 |
| 23.2 | 99 |
| 25.0 | 14 |
| 26.5 | 32 |
| 28.8 | 35 |
| 32.3 | 15 |

ss ¹³C NMR peak list for the crystalline form, containing acetone solvent is shown in Table 14.

TABLE 14

| ¹³C Chemical | |
|---|---|
| Shifts [ppm]^{a} | Intensity^{b} |
| 165.8 | 5.58 |
| 157.1 | 3.74 |
| 152.2 | 4.69 |
| 151.0 | 6.35 |
| 122.5 | 3.46 |
| 120.6 | 2.58 |
| 119.6 | 1.28 |
| 102.5 | 7.84 |
| 99.7 | 5.51 |
| 56.7 | 5.55 |
| 45.0 | 5.08 |
| 38.0 | 4.34 |
| 32.7 | 9.95 |
| 32.2 | 12.00 |
| 31.7 | 9.23 |
| 31.3 | 8.88 |
| 12.0 | 3.94 |
| 11.0 | 5.43 |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.

ss ¹³C NMR peak list for the crystalline form, containing 1-butanol and ethanol solvents is shown in Table 15.

TABLE 15

| ¹³C Chemical | |
|---|---|
| Shifts [ppm]^{a} | Intensity^{b} |
| 165.8 | 4.56 |
| 156.9 | 3.96 |
| 152.2 | 5.00 |
| 151.0 | 5.28 |
| 122.2 | 3.73 |
| 120.6 | 2.41 |
| 119.6 | 1.13 |
| 102.4 | 6.23 |
| 99.7 | 6.29 |
| 60.1 | 0.98 |
| 56.9* | Peak shoulder |
| 56.5 | 6.45 |
| 44.9 | 5.09 |
| 37.7 | 4.84 |
| 33.8 | 1.49 |
| 32.6 | 9.51 |
| 31.5 | 12.00 |
| 19.2 | 0.48 |
| 18.8 | 1.25 |
| 15.3 | 0.50 |
| 11.0 | 4.36 |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.
*Peak shoulder

ss ¹³C NMR peak list for the crystalline form, containing N,N-dimethylformamide (DMF) solvent is shown in Table 16.

TABLE 16

| ¹³C Chemical | |
|---|---|
| Shifts [ppm]^{a} | Intensity^{b} |
| 165.9 | 4.80 |
| 162.1 | 0.93 |
| 156.8 | 3.19 |
| 152.3 | 4.08 |
| 150.9 | 5.16 |
| 122.2 | 4.04 |
| 120.5 | 2.23 |
| 119.5 | 0.98 |
| 102.5 | 5.97 |
| 99.8 | 7.04 |
| 63.1 | 0.25 |
| 56.5 | 5.74 |
| 44.9 | 4.56 |
| 37.6 | 4.43 |
| 37.1* | Peak shoulder |
| 32.6 | 9.14 |
| 31.8 | 12.00 |
| 31.0 | 6.10 |
| 29.5 | 0.40 |
| 27.5 | 0.15 |
| 27.2 | 0.21 |
| 11.0* | Peak shoulder |
| 10.4 | 7.12 |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.
*Peak shoulder

ss ¹³C NMR peak list for the crystalline form, containing tetrahydrofuran (THF) solvent is shown in Table 17.

TABLE 17

| ¹³C Chemical | |
|---|---|
| Shifts [ppm]^{a} | Intensity^{b} |
| 165.9 | 4.97 |
| 156.9 | 3.41 |
| 152.2 | 4.62 |
| 151.0 | 5.98 |
| 122.3 | 4.36 |
| 120.6 | 2.60 |
| 119.6 | 1.25 |
| 102.4 | 6.37 |
| 99.7 | 6.55 |
| 68.6 | 1.65 |
| 63.1 | 1.50 |
| 56.5 | 6.42 |
| 44.9 | 5.37 |
| 37.7 | 5.03 |
| 32.6 | 9.80 |
| 32.0 | 10.20 |
| 31.5 | 12.00 |
| 31.3* | Peak shoulder |
| 27.7 | 1.08 |
| 27.2 | 0.79 |
| 26.2 | 1.62 |
| 11.7* | Peak shoulder |
| 11.3 | 6.84 |
| 10.8* | Peak shoulder |
| 10.2 | 1.41 |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.
*Peak shoulder

Glass transition temperature of the non-crystalline form is shown in Table 18.

TABLE 18

| | Glass Transition |
|---|---|
| | Temperature |
| Sample | (Midpoint) |
| Non-crystalline | 87° C. |

ss ¹³C NMR peak list for the non-crystalline form is shown in Table 19.

TABLE 19

| ¹³C Chemical | |
|---|---|
| Shifts [ppm]^{a} | Intensity^{b} |
| 161.9 | 5.66 |
| 157.7 | 6.49 |
| 152.0 | 11.72 |
| 121.1 | 3.49 |
| 116.8 | 3.33 |
| 103.3 | 12.00 |
| 54.7 | 2.85 |
| 46.3 | 2.47 |
| 41.1 | 4.92 |
| 31.8 | 10.78 |
| 26.0 | 5.63 |
| 17.5 | 2.10 |
| 12.2 | 4.73 |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.

Raman peak list for the non-crystalline form is shown in Table 20.

TABLE 20

| Wavenumber | Relative |
|---|---|
| (cm⁻¹) | Intensity |
| 159 | Weak |
| 169 | Weak |
| 177 | Weak |
| 185 | Weak |
| 209 | Weak |
| 218 | Weak |
| 223 | Weak |
| 252 | Weak |
| 260 | Weak |
| 267 | Weak |
| 276 | Weak |
| 281 | Weak |
| 285 | Weak |
| 307 | Weak |
| 313 | Weak |
| 316 | Weak |
| 325 | Weak |
| 328 | Weak |
| 342 | Weak |
| 350 | Weak |
| 355 | Weak |
| 366 | Weak |
| 369 | Weak |
| 391 | Weak |
| 421 | Weak |
| 430 | Weak |
| 437 | Weak |
| 448 | Weak |
| 455 | Weak |
| 461 | Weak |
| 466 | Weak |
| 476 | Weak |
| 479 | Weak |
| 483 | Weak |
| 488 | Weak |
| 494 | Weak |
| 507 | Weak |
| 511 | Weak |
| 527 | Weak |
| 546 | Weak |
| 551 | Weak |
| 553 | Weak |
| 571 | Weak |
| 581 | Weak |
| 587 | Weak |
| 606 | Weak |
| 642 | Weak |
| 644 | Weak |
| 669 | Weak |
| 685 | Weak |
| 687 | Weak |
| 691 | Weak |
| 697 | Weak |
| 712 | Weak |
| 730 | Weak |
| 733 | Weak |
| 772 | Weak |
| 797 | Weak |
| 800 | Weak |
| 827 | Weak |
| 853 | Weak |
| 866 | Weak |
| 872 | Weak |
| 883 | Weak |
| 888 | Weak |
| 891 | Weak |
| 907 | Weak |
| 925 | Weak |
| 949 | Weak |
| 961 | Weak |
| 994 | Weak |
| 1009 | Weak |
| 1026 | Weak |
| 1055 | Weak |
| 1058 | Weak |
| 1067 | Weak |
| 1080 | Weak |
| 1100 | Weak |
| 1111 | Weak |
| 1125 | Weak |
| 1140 | Weak |
| 1175 | Weak |
| 1193 | Weak |
| 1231 | Weak |
| 1280 | Medium |
| 1311 | Strong |
| 1342 | Weak |
| 1410 | Weak |
| 1414 | Weak |
| 1453 | Medium |
| 1456 | Medium |
| 1493 | Medium |
| 1506 | Strong |
| 1562 | Weak |
| 1577 | Weak |
| 1655 | Weak |
| 2258 | Weak |
| 2735 | Weak |
| 2814 | Weak |
| 2882 | Weak |
| 2927 | Medium |
| 3002 | Weak |
| 3121 | Weak |
| 3143 | Weak |
| 3179 | Weak |

FT-IR peak list for the non-crystalline form is shown in Table 21.

TABLE 21

| Wavenumber | Relative |
|---|---|
| (cm⁻¹) | Intensity |
| 574 | Medium |
| 578 | Medium |
| 605 | Medium |
| 642 | Weak |
| 667 | Weak |
| 683 | Weak |
| 721 | Medium |
| 770 | Weak |
| 796 | Medium |
| 827 | Weak |
| 883 | Medium |
| 903 | Medium |
| 925 | Medium |
| 960 | Weak |
| 979 | Weak |
| 998 | Weak |
| 1022 | Weak |
| 1046 | Medium |
| 1097 | Weak |
| 1107 | Weak |
| 1135 | Weak |
| 1174 | Weak |
| 1226 | Medium |
| 1252 | Medium |
| 1305 | Medium |
| 1340 | Medium |
| 1407 | Strong |
| 1451 | Medium |
| 1468 | Medium |
| 1487 | Medium |
| 1507 | Weak |
| 1554 | Strong |
| 1647 | Medium |
| 1896 | Weak |
| 2451 | Weak |
| 2701 | Weak |
| 2751 | Weak |
| 2859 | Weak |
| 2927 | Weak |
| 2958 | Weak |
| 3113 | Weak |
| 3191 | Weak |

Solvent levels for the crystalline form isolated by process 1 are shown in Table 22.

TABLE 22

| Form A | 2-propanol | Water |
|---|---|---|
| (Batch #) | (% Weight) | (% Weight) |
| 1 | 2.6 | 1.0 |
| 2 | 2.9 | 0.8 |

Solvent levels for the crystalline form isolated by process 2 are shown in Table 23.

TABLE 23

| Form A | 2-propanol | Water |
|---|---|---|
| (Batch #) | (% Weight) | (% Weight) |
| 1 | 2.8 | 0.9 |
| 2 | 2.8 | 2.6 |

Solvent levels for the crystalline form isolated by multiple processes are shown in Table 24.

TABLE 24

| Form A | 2-propanol | Water | Isolation |
|---|---|---|---|
| (Batch #) | (% Weight) | (% Weight) | Process |
| 1 | Not Measured | 4.0 | Process 4 |
| 2 | 0.0 | 1.9 | Process 5 |
| 3 | 2.8 | 0.9 | Process 2 |
| 4 | Not Measured | 0.5 | Process 6 |
| 5 | 4.7 | Not Measured | Process 3 |

Crystallographic data for the crystalline form at 23° C. is shown in Table 25.

TABLE 25

| Empirical Formula | C₁₆H₂₀N₆O•H₂O |
|---|---|
| Formula Weight | 330.40 |
| Crystal System | Tetragonal |
| Space Group | I4 |
| Unit Cell | a = 19.6 Å |
| Dimensions | b = 19.6 Å |
| | c = 8.7 Å |
| | α = 90.0° |
| | β = 90.0° |
| | γ = 90.0° |
| Z | 8 |

Crystallographic data for the crystalline form at 120° C. is shown in Table 26.

TABLE 26

| Empirical Formula | C₁₆H₂₀N₆O |
|---|---|
| Formula Weight | 312.38 |
| Crystal System | Tetragonal |
| Space Group | I4 |
| Unit Cell | a = 19.7 Å |
| Dimensions | b = 19.7 Å |
| | c = 8.7 Å |
| | α = 90.0° |
| | β = 90.0° |
| | γ = 90.0° |
| Z | 8 |

The present invention also provides pharmaceutical compositions comprising a crystalline or non-crystalline form, and to methods for preparing such forms, as well as pharmaceutical compositions for use in medicine and for use in treating such diseases as psoriasis and dermatitis. The present invention also provides the use of such pharmaceutical compositions in the manufacture of a medicament for treating such diseases as psoriasis and dermatitis

Methods of treating the diseases and syndromes listed herein are understood to involve administering to an individual in need of such treatment a therapeutically effective amount of the polymorph of the invention, or a composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, goats, horses, or primates, and most preferably humans. As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

### Dosage and Formulation

The invention also includes pharmaceutical compositions utilizing one or more of the present polymorphs along with one or more pharmaceutically acceptable carriers, excipients, vehicles, etc.

Topical formulations of the presently disclosed polymorph of crystalline form or a non-crystalline form of 3-((3R,4R)-4-methyl-3-[methyl-(7H-pyrrolo[2,3-d]pyrimidin-4-yl)-amino]-piperidin-1-yl)-3-oxo-propionitrile may be administered topically, (intra)dermally, or transdermally to the skin or mucosa. Topical administration using such preparations encompasses all conventional methods of administration across the surface of the body and the inner linings of body passages including epithelial and mucosal tissues, including transdermal, epidermal, buccal, pulmonary, ophthalmic, intranasal, vaginal and rectal modes of administration. Typical formulations for this purpose include gels, hydrogels, lotions, solutions, creams, colloid, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Such topical formulations may be prepared in combination with additional pharmaceutically acceptable excipients. An excipient which has been determined to be essential to clinical efficacy is one or more penetration enhancer such as be one or more saturated or cis-unsaturated C10-C18 fatty alcohols. Preferably, such fatty alcohols include C16-C18 fatty alcohols, and most preferably, are a C18 fatty alcohol. Examples of cis-unsaturated C16-C18 fatty alcohols include oleyl alcohol, linoleyl alcohol, γ-linolenyl alcohol and linolenyl alcohol. Oleyl alcohol is most preferred as a penetration enhancer. Saturated C10-C18 fatty alcohols useful as penetration enhancers include decyl alcohol, lauryl alcohol, myristyl alcohol, cetyl alcohol and stearyl alcohol. Alternatively, other penetration enhancers which may be used to prepare the topical formulations include C10-C18 fatty acids, which when saturated may include capric acid, lauric acid, myristic acid, palmitic acid, stearic acid and arachidic acid. Preferably, the penetration enhancer may be a C16-C18 fatty acid, and more preferably, a C18 fatty acid. Alternatively, the penetration enhancer may usefully be a cis-unsaturated fatty acid, such as palmitoleic acid (cis-9-hexadecenoic acid), oleic acid (cis-9-octadecenoic acid), cis-vaccenic acid (cis-11-octadecenoic acid), linoleic acid (cis-9,12-octadecadienoic acid), γ-linolenic acid (cis-6,9,12-octadecatrienoic acid), linolenic acid (cis-9,12,15-octadecatrienoic acid) and arachidonic acid (cis-5,8,11,14-eicosatetraenoic acid). The penetration enhancers, for example, one selected from C10-C18 fatty alcohols, are used in amounts ranging from about 0.1 to about 5% (w/v), more preferably, from 1 to about 4%, more preferably still, 1 to about 3%, and, most preferably, about 2.0% (w/v). In general, any penetration enhancer or combination thereof may be included in PEG-based Ointment formulations that are able to achieve percutaneous flux at a level equal to or greater than achieved by formulations containing about 2% oleyl alcohol.

Topical formulations contain 3-((3R,4R)-4-methyl-3-[methyl-(7H-pyrrolo[2,3-d]pyrimidin-4-yl)amino]-piperidin-1-yl)-3-oxopropionitrile in therapeutically effective amounts that can be given in daily or twice daily doses to patients in need. These amounts range from about 0.1% to about 5.0% (w/v), more preferably, from about 0.1% to about 3.0%, more preferably still, from about 0.5% to about 2.3%, and, most preferably, about 2.0% (w/v). Among other excipients which enhance the stability of these formulations include aldehyde scavengers, such as glycerine and propylene glycol, and antioxidants, such as butyl hydroxyanisole (BHA), butyl hydroxytoluene (BHT), propyl gallate, ascorbic acid (Vitamin C), polyphenols, tocopherols (Vitamin E), and their derivatives. Preferably, PEG-based ointment formulations containing at least 30% polyethylene glycol, tofacifinib and one or more penetration enhancers and additional pharmaceutically acceptable excipients forming a stable formulation such that the level of total degradants is no more than 7% when the product is stored at 40° C. for 4 weeks. More preferably, addition of aldehyde scavengers and antioxidants into formulations Ointment 1 (A) and Ointment 2 (C) stabilized the polyethylene glycol containing ointment formulations such that the level of total degradants is no more than 5% when the product is stored at 40° C. for 4 weeks.

The invention further provides a pharmaceutical composition as set forth above, wherein the pharmaceutically acceptable carrier is at least 30% by weight PEG, and further comprising stabilizing excipients in an amount sufficient to achieve a chemically stable formulation such that the level of total degradants is not more that 7% by weight after 4 weeks at 40° C.

The invention also provides a pharmaceutical composition as set forth above, wherein the pharmaceutically acceptable carrier is at least 30% by weight PEG, and further comprising one or more aldehyde scavenger or anti-oxidant excipient in an amount sufficient to achieve a chemically stable formulation such that the level of total degradants is not more that 7% by weight after 4 weeks at 40° C.

The invention further provides a pharmaceutical composition as set forth above which is characterized by having a percutaneous flux measured by in vitro methods known in the art that is equal or greater than the flux measured from a composition consisting by weight of about 2% tofacitinib free base, about 1.8% oleyl alcohol, about 17.9% glycerine, about 18% propylene glycol, about 30% PEG 400, about 30% PEG 3350, and about 0.1% BHA.

The compounds of these teachings can be prepared by methods known in the art. The reagents used in the preparation of the compounds of these teachings can be either commercially obtained or can be prepared by standard procedures described in the literature. For example, compounds of the present invention can be prepared according to the methods illustrated in the following examples.

The description of this invention utilizes a variety of abbreviations well known to those skilled in the art, including the following:

aq.: aqueous

### CH₃CN: Acetonitrile

### DCM: Dichloromethane

### DMF: N,N-Dimethylformamide

### DMSO: Dimethylsulfoxide

EtOAc: Ethyl acetate

### EtOH: Ethanol

### FT-IR: Fourier Transform-Infrared

HOAc: Acetic acid

### MeOH: Methanol

PXRD: powder X-ray diffraction
ss ¹³C NMR: solid state ¹³C nuclear magnetic resonance

### THF: Tetrahydrofuran

### TLC: Thin Layer Chromatography

### EXAMPLES

The following non-limiting examples are presented merely to illustrate the present invention. The skilled person will understand that there are numerous equivalents and variations not exemplified but which still form part of the present teachings.

### Preparation of 3-((3R,4R)-4-methyl-3-[methyl-(7H-pyrrolo[2,3-d]pyrimidin-4-yl)-amino]-piperidin-1-yl)-3-oxopropionitrile Solid Forms

### Example 1

2-Propanolate (Process 1): The crystalline form was prepared by adding 750 grams of the citrate salt of 3-((3R,4R)-4-methyl-3-[methyl-(7H-pyrrolo[2,3-d]pyrimidin-4-yl)-amino]-piperidin-1-yl)-3-oxopropionitrile to mixture of 2-propanol (3.8 L) and water (3.8 L). The resulting mixture was stirred for approximately 1 hour at 20° C. Four liters of 1 molar sodium hydroxide aqueous solution were then added to the mixture over 40 minutes. The mixture was then stirred at 20° C. for approximately 17 hours. Solids were isolated by vacuum filtration, washed twice with 1.9 L of water, and dried under reduced pressure at 65° C. for approximately 30 hours. The resulting crystalline solids contained 1.0% weight water by Karl Fischer analysis and 2.6% weight 2-propanol by residual solvent analysis.

### Example 2

2-Propanolate (Process 2): The crystalline form was prepared by adding 271 grams of the citrate salt of 3-((3R,4R)-4-methyl-3-[methyl-(7H-pyrrolo[2,3-d]pyrimidin-4-yl)-amino]-piperidin-1-yl)-3-oxopropionitrile to 2-propanol (1.36 L)/water (1.36 L) (1:1, by volume) solvent system at room temperature. Mixing was facilitated with an overhead stirrer throughout the experiment. While providing high agitation to the slurry, 1.88 L of 1.0N sodium hydroxide aqueous solution was slowly added at 20° C. A 1% weight crystalline form crystalline seed was then added to the reactor and allowed to stir several hours at ambient temperature resulting in a slurry. The solids were isolated by vacuum filtration, washed with water and dried under reduced pressure at 60° C. to 70° C. The resulting crystalline solids contained 0.9% weight water and 2.8% weight 2-propanol, as determined through Karl Fischer and residual solvent analyses, respectively.

### Example 3

2-Propanolate (Process 3): The crystalline form was prepared by adding 218 mg of non-crystalline 3-((3R,4R)-4-methyl-3-[methyl-(7H-pyrrolo[2,3-d]pyrimidin-4-yl)-amino]-piperidin-1-yl)-3-oxopropionitrile to 0.5 mL of 2-propanol. The mixture was stirred for approximately 5 days at room temperature, isolated by vacuum filtration and dried under reduced pressure at 70° C. for 1 day. The resulting crystalline solids contained 4.7% weight 2-propanol by residual solvent analysis.

### Example 4

Methanolate (Process 4): The crystalline form was prepared by adding 518 mg non-crystalline 3-((3R,4R)-4-methyl-3-[methyl-(7H-pyrrolo[2,3-d]pyrimidin-4-yl)-amino]-piperidin-1-yl)-3-oxopropionitrile to 25 mL of methanol/water (1:3, by volume) solvent system at room temperature. Mixing was facilitated with a magnetic stir bar throughout the experiment. The mixture was then heated at 1.9° C./minute to 50° C. The suspension was maintained at 50° C. for 5 minutes, cooled at 1.0° C./minute to 5° C., and slurried at 5° C. for 75 minutes. Solids were isolated by vacuum filtration and dried under ambient conditions for approximately 19 hours. Approximately 0.6% weight methanol and 4.0% weight water were detected within the resulting crystalline form solids by thermogravimetric analysis with IR detection of the evolved gases.

### Example 5

Acetone Solvate (Process 5): The crystalline form was prepared by dissolving 130 grams of 3-((3R,4R)-4-methyl-3-[methyl-(7H-pyrrolo[2,3-d]-pyrimidin-4-yl)-amino]-piperidin-1-yl)-3-oxopropionitrile in 1.5 L of an acetone/water mixture (75% acetone, by volume) at 54° C. The mixture was then cooled quickly to 25° C., maintained at 25° C. for 3 hours, and then cooled to 5° C. Solids were isolated by vacuum filtration and dried under reduced pressure at 50° C. for approximately 17 hours. The resulting crystalline solids contained 1.9% weight water by Karl Fischer analysis and 0.6% weight acetone by residual solvent analysis.

### Example 6

n-Butanolate/ethanolate (Process 6): Methyl-[(3R,4R)-4-methyl-piperidin-3-yl]-(7H-pyrrolo[2,3-d]pyrimidin-4-yl)-amine was prepared as described in example 10 of WO 2007/012953. A solution of 1.33 g of methyl-[(3R,4R)-4-methyl-piperidin-3-yl]-(7H-pyrrolo[2,3-d]pyrimidin-4-yl)-amine was added to 5 mL of 1-butanol in a round bottom flask. To this same flask was added 0.41 mL of 1,8-diazabicyclo[5.4.0]undec-7-ene (417 mg, 0.5 eq.) followed by 1.15 mL of ethyl cyanoacetate (1218 mg, 2.0 eq.). The mixture was stirred under a nitrogen atmosphere. The mixture was heated to 40° C. and allowed to stir at this temperature for 17 hours. The resulting suspension was cooled at 1° C./min. to 20° C., and slurried at 20° C. for approximately 48 hours. Solids were isolated by vacuum filtration, washed with 50 mL of 1-butanol followed by 50 mL of acetone and dried under vacuum at 55° C. for approximately 18 hours. The resulting crystalline form solids contained 0.5% weight water by Karl Fischer analysis, and 2.7% weight n-butanol, 0.2% weight acetone and 1.8% weight ethanol by residual solvent analysis.

### Example 7

N,N-Dimethylformamide Solvate (Process 7): The crystalline form was prepared by adding 614 mg of the crystalline form prepared form process 1 to 12 mL of N,N-dimethylformamide/methyl tert-butyl ether (1:5, by volume) solvent system at room temperature. Mixing was facilitated with a magnetic stir bar throughout the experiment. The mixture was then heated to between 40-50° C. and cooled to room temperature eight times over 13 days. Solids were isolated from the mixture by vacuum filtration and dried at 70° C. under reduced pressure for 1 day. The presence of N,N-dimethylformamide within the resulting crystalline form was demonstrated by ¹³C CPMAS solid-state NMR spectroscopy.

### Example 8

Tetrahydrofuran Solvate (Process 8): The crystalline form was prepared by adding 633 mg of the crystalline form prepared by process 1 to 10 mL of tetrahydrofuran/heptane (2:1, by volume) solvent system at room temperature. Mixing was facilitated with a magnetic stir bar throughout the experiment. The mixture was then heated to between 40-50° C. and cooled to room temperature eight times over 13 days. Solids were isolated from the mixture by vacuum filtration and dried at 70° C. under reduced pressure for one day. The presence of tetrahydrofuran within the resulting crystalline form was demonstrated by ¹³C CPMAS solid-state NMR spectroscopy.

### Example 9

Hydrate (Process 9): Crystalline form was prepared by evaporation of an 18 mg/mL 3-((3R,4R)-4-methyl-3-[methyl-(7H-pyrrolo[2,3-d]pyrimidin-4-yl)-amino]-piperidin-1-yl)-3-oxopropionitrile solution in 1,4-dioxane/water (1:1, by volume) at 50° C.

### Example 10

Non-crystalline form was prepared by suspending 40 grams of the citrate salt of 3-((3R,4R)-4-methyl-3-[methyl-(7H-pyrrolo[2,3-d]pyrimidin-4-yl)-amino]-piperidin-1-yl)-3-oxopropionitrile in 400 mL of a water/n-butanol (50% v/v). 32.9 grams of potassium carbonate (K₂CO₃) was added to the suspension and allowed to equilibrate for 15 minutes. A separatory funnel was then utilized to isolate the organic layer within the mixture, wash the isolated organic layer with 200 mL of water, and isolate the resulting washed organic layer. The washed organic layer was filtered into a 500 mL round bottom flask. The washed organic layer was concentrated by rotary evaporation with a bath temperature of 60° C. to produce a solid. 150 mL of toluene was then added to the resulting solid and the mixture concentrated again by rotary evaporation with a bath temperature of 60° C. to produce a thick solution. 150 mL of toluene was then added to the resulting solution and again concentrated to produce a solid. 150 mL of acetonitrile was then added to the resulting solid and the mixture concentrated rotary evaporation. The resulting product was then placed under reduced pressure for approximately 17 hours to yield 23.2 gms of the non-crystalline material.

### Example 11

Non-crystalline form was prepared by mixing 2.1 gms of crystalline form in 200 mL of acetone at room temperature for 1 day. The suspension was filtered at room temperature to produce a clear solution. The solvent was then evaporated from the solution using a BUCHI Rotovapor R-205 (BUCHI Labortechnik AG, Switzerland), an Edwards RV3 vacuum pump (West Sussex, United Kingdom), and a BUCHI heating bath B-490 (BUCHI Labortechnik AG, Switzerland) maintained at 40° C. to isolate an amorphous material. The isolated amorphous material was dried under vacuum at 40° C. for 1 day, followed by 80° C. for 4 days and 100° C. for 1 day to yield non-crystalline material.

Variations, modifications, and other implementations of what is described herein will occur to those skilled in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced therein.

Each of the printed publications, including but not limited to patents, patent applications, books, technical papers, trade publications and journal articles described or referenced in this specification are herein incorporated by reference in their entirety and for all purposes.

### Example 12

A randomized, double-blind, vehicle-controlled, four-arm, parallel-group study was carried out to characterize the efficacy of two topical formulations of tofacitinib (also known as tasocitinib or CP-690,550) free base (2%) administered BID (twice daily) for 4 weeks in subjects with chronic mild to moderate plaque psoriasis.

A total of 71 subjects were enrolled (approximately 24 subjects per drug group and 12 subjects per vehicle group) in order to yield 68 completed subjects. Subjects were randomized to 1 of 4 treatment groups in the ratio of 2:1:2:1 (Table 27). To make the ointment, the ingredients listed in Table 28 are added into suitable vessels with continuous agitation, and heated to approximately 65° C. in order to melt the PEG3350. Once PEG3350 is completely melted, while being agitated, the mixture is cooled to below 40° C. to initiate congealing. The congealed semi-solid mass is then filled into individual tubes suitable for dispensing.

TABLE 27

| | Formulation | |
|---|---|---|
| | Designation | |
| Treatment Group A | Ointment 1 | 2% tofacitinib Ointment 1 BID |
| Treatment Group B | Vehicle 1 | Vehicle 1 BID |
| Treatment Group C | Ointment 2 | 2% tofacitinib Ointment 2 BID |
| Treatment Group D | Vehicle 2 | Vehicle 2 BID |

Ointment 1 and Vehicle 1 contained oleyl alcohol at 2%, whereas Ointment 2 and Vehicle 2 did not contain oleyl alcohol. The compositions of the formulations administered to the four test groups are shown in Table 28.

TABLE 28
PEG-based ointment formulation compositions
used in clinical study.

| | Formulation Designation | | | |
|---|---|---|---|---|
| | Ointment 1 | Vehicle 1 | Ointment 2 | Vehicle 2 |
| | (A) | (B) | (C) | (D) |
| Tofacitinib free | 2.0 | 0.0 | 2.0 | 0.0 |
| base | | | | |
| Oleyl Alcohol | 2.0 | 2.0 | 0.0 | 0.0 |
| Glycerin | 17.9 | 19.9 | 17.9 | 19.9 |
| Propylene Glycol | 18.0 | 18.0 | 20.0 | 20.0 |
| Polyethylene | 30.0 | 30.0 | 30.0 | 30.0 |
| Glycol 400 | | | | |
| Polyethylene | 30.0 | 30.0 | 30.0 | 30.0 |
| Glycol 3350* | | | | |
| Butyl Hydroxy- | 0.1 | 0.1 | 0.1 | 0.1 |
| anisole (BHA) | | | | |
| Total | 100.0 | 100.0 | 100.0 | 100.0 |
| (weight/weight) | | | | |

*Polyethylene Glycol 3350 contains 100 ppm of Butylated Hydroxytoluene (BHT)

Treatments were applied to the treatment area topically BID for 4 weeks at an application coverage of approximately 3 mg/cm². Study drug total treatment area size was fixed at a single 300 cm² (˜1.5% BSA) area, which may have included all of or a portion of one or more psoriatic plaques. One of the plaques was identified as the target plaque, which had to be at least 9 cm² in size. If the selected treatment area includes normal skin in addition to psoriasis plaques, study drug was also applied to the normal (peri-lesional) skin in the treatment area.A target plaque was selected at Baseline and evaluated for Target Plaque Severity Score (TPSS). This assessment was performed on all subsequent visits to evaluate efficacy. Plaques that were intertriginous or on the hands, feet, neck, face, elbows, knees, below the knees, and scalp were deemed not eligible to be target plaques or to be included in the treatment area.Active treatment (Ointment 1 or Ointment 2), or vehicle (Vehicle 1 or Vehicle 2) was applied to the treatment area according to a BID dosing regimen. Pharmacokinetic (PK) sampling was done at Week 4 at pre-dose (0 hour) and post-dose at 1, 2, and at any time-point between 4-9 hours. The target plaque was scored individually by the investigator (or a properly trained evaluator) for signs of induration, scaling, and erythema. Each of the 3 signs was rated on a 5-point (0-4) severity scale (Table 29).

TABLE 129
Component Scoring Criteria for the
Target Plaque Severity Score (TPSS)

| | | |
|---|---|---|
| Score | Label | Description |

Erythema (E)

| | | |
|---|---|---|
| 0 | None | No evidence of erythema (post-inflammatory |
| | | hyperpigmentation and/or hypopigmentation may be |
| | | present) |
| 1 | Slight | Light pink |
| 2 | Moderate | Light red |
| 3 | Marked | Red |
| 4 | Very Marked | Dark, deep red |

Induration (I)

| | | |
|---|---|---|
| 0 | None | No evidence of plaque elevation |
| 1 | Slight | Barely palpable |
| 2 | Moderate | Slight, but definite elevation, indistinct edges |
| 3 | Marked | Elevated with distinct edges |
| 4 | Very Marked | Marked plaque elevation, hard/sharp borders |

Scaling (S)

| | | |
|---|---|---|
| 0 | None | No evidence of scaling |
| 1 | Slight | Occasional fine scale |
| 2 | Moderate | Fine scale predominates |
| 3 | Marked | Coarse scale predominates |
| 4 | Very Marked | Thick, coarse scale predominates |

The individual signs severity subscores are summed (E+I+S). The TPSS can vary in increments of 1 and range from 0 to 12, with higher scores representing greater severity of psoriasis. For the primary efficacy endpoint, statistical significance was claimed if the upper limit of the one-sided 90% confidence limit (of the difference between tofacitinib ointment and the vehicle) is less than 0. The study showed statistically significant evidence of efficacy for the contrast tofacitinib Ointment 1 (A)—Vehicle 1 (B) based on percent change from Baseline in TPSS at Week 4. Contrast tofacitinib Ointment 2 (C)—Vehicle 2 (D) did not achieve statistical significance. Descriptive statistics for the TPSS at Baseline and Week 4 for the Full Analysis Set (FAS) are presented in Table 30. Mean TPSS scores at Baseline ranged from 6.80 (tofacitinib Ointment 2) to 7.31 (Vehicle 1) and at Week 4 ranged from 3.55 (tofacitinib Ointment 1) to 5.89 (Vehicle 2) across the treatment groups. Among the 4 treatment groups, tofacitinib Ointment 1 (which contained oleyl alcohol) had the largest mean and mean percent decreases from Baseline (changes of −3.73 and −53.97%, respectively), while Vehicle 2 had the smallest mean and mean percent decreases from Baseline (changes of −1.22 and −17.24%, respectively). The primary analysis was the LSmean difference between tofacitinib and vehicle (i.e., tofacitinib Ointment 1 vs. Vehicle 1 [Contrast 1] and tofacitinib Ointment 2 vs. Vehicle 2 [Contrast 2]) for the percent change from Baseline in TPSS at Week 4 for the FAS (Table 31). The LSmean difference for Contrast 1 (CP-690,550 Ointment 1 minus Vehicle 1) was −12.87% and the 1-sided 90% upper CL was −0.71% (significant). The LSmean difference for Contrast 2 (CP-690,550 Ointment 2 minus Vehicle 2) was −6.97% and the 1-sided 90% upper CL was 6.62% (nonsignificant). In addition, 13% of tofacitinib Ointment 1 subjects has complete clearing of their target plaque, whereas no subjects applying Vehicle 1, tofacitinib Ointment 2, or Vehicle 2 had complete clearing. PK data were available from 44 subjects treated with 2% tofacitinib ointment. Sixty-two percent (62%, 13/21) of subjects on tofacitinib Ointment 1 had at-least one time-point with quantifiable tofacitinib concentration (above the lower limit of quantification [LLOQ], 0.1 ng/mL) compared to 26% (6/23) of subjects on tofacitinib Ointment 2. The maximum observed concentration was 0.96 and 0.65 ng/mL on tofacitinib Ointment 1 and tofacitinib Ointment 2, respectively.

TABLE 30
Summary of Descriptive Statistics for TPSS at Week 4 (FAS, No Imputation)

| | 2% tofacitinib | | 2% tofacitinib | |
|---|---|---|---|---|
| | Ointment 1 | Vehicle 1 | Ointment 2 | Vehicle 2 |
| | BID | BID | BID | BID |
| Baseline | | | | |
| N | 23 | 13 | 25 | 10 |
| Mean (SD) | 7.22 (1.51) | 7.31 (1.38) | 6.80 (1.19) | 7.20 (1.40) |
| Week 4 | | | | |
| N | 22 | 13 | 23 | 9 |
| Mean (SD) | 3.55 (2.67) | 4.23 (1.48) | 5.09 (2.48) | 5.89 (1.96) |
| Mean Chg (SD) | −3.73 (1.98) | −3.08 (1.75) | −1.65 (2.37) | −1.22 (1.56) |
| Mean % Chg (SD) | −53.97 (29.93) | −41.01 (20.29) | −24.26 (33.43) | −17.24 (25.40) |

Abbreviations: BID = twice daily; N = Number of subjects; SD = standard deviation; Mean Chg = mean change from Baseline; Mean % Chg = mean percent change from Baseline; TPSS = Target Plaque Severity Score; FAS = Full Analysis Set 0 = no involvement, 1 = slight, 2 = moderate, 3 = marked, and 4 = very marked.

TABLE 31
Statistical Analysis of Percent Change From Baseline in TPSS at Week 4 (Longitudinal
Model), Contrast 1 (A-B) and Contrast 2 (C-D) (FAS, No Imputation)

| | | | Difference from Vehicle^{a} | | |
|---|---|---|---|---|---|
| | | | | 1-Sided 90% | |
| | | | | Confidence Limit^{b} | |
| Treatment | N | LS Mean (SE) | Diff^{c} (SE) | Lower | Upper |
| 2% tofacitinib | 22 | −54.40 (10.71) | −12.87 (9.42) | −25.03 | −0.71* |
| Ointment 1 BID | | | | | |
| Vehicle 1 BID | 13 | −41.53 (11.73) | | | |
| 2% tofacitinib | 23 | −24.19 (10.58) | −6.97 (10.53) | −20.57 | 6.62 |
| Ointment 2 BID | | | | | |
| Vehicle 2 BID | 9 | −17.21 (12.73) | | | |

Abbreviations: N = number of subjects; BID = twice daily; SE = standard error; TPSS = Target Plaque Severity Score; FAS = Full Analysis Set; LS = least squares; CI = confidence interval
Ointment 1 and Vehicle 1 contained oleyl alcohol, whereas Ointment 2 and Vehicle 2 did not.
Results are obtained from a longitudinal mixed-effect model with percent change from Baseline as the response.
The effects of treatment, week, and treatment-by-week interaction are included as fixed effects, along with subject as a random effect and Baseline as a covariate.
*Statistically significant.
^{a}Contrast 1(A-B) = 2% tofacitinib Ointment 1 BID minus Vehicle 1. Contrast 2(C-D) = 2% tofacitinib Ointment 2 BID minus Vehicle 2.
^{b}One-sided 90% upper and lower confidence limits represent 2-sided 80% CI.
^{c}Difference = (tofacitinib Ointment − Vehicle).

### Example 13

PEG-based Ointment formulations containing 3 different penetration enhancers (oleyl alcohol, Span 80, or glycerol monooleate) were tested for in vitro percutaneous skin absorption. Based on in vitro percutaneous absorption testing (using two separate skin donors) tofacitinib PEG-based ointment formulation containing 1.8% oleyl alcohol showed a significant increase in both cumulative permeation and flux. No significant increase was observed for the formulations containing 1.9% Span 80, and 2.1% glycerol monooleate (GM). The ointment composition with 1.8% oleyl alcohol is similar to Ointment 1 in Table 28.

TABLE 32
Tofacitinib Flux through Human Cadaver Skin with three PEG-based
ointment formulations

| | | | | | PEG- | | | |
|---|---|---|---|---|---|---|---|---|
| | | | PEG-based | | based | | | |
| | | | Ointment | | Ointment | | PEG-based | |
| | PEG- | | with | | with | | Ointment | |
| | based | | 1.8% | | 1.9% | | with | |
| | Ointment | | OA | | Span 80 | | 2.1% GM | |
| | Donor | | | | | | | |
| Formulation | 1 | 2 | 1 | 2 | 1 | 2 | 1 | 2 |
| Flux, | 0.5 | 0.0 | 0.7 | 0.7 | 0.1 | 0.0 | 0.2 | 0.0 |
| μg/cm²/hr @ | | | | | | | | |
| 24 hrs | | | | | | | | |
| Flux, | 1.6 | 0.1 | 2.3 | 2.0 | 0.9 | 0.2 | 1.8 | 0.2 |
| μg/cm²/hr @ | | | | | | | | |
| 48 hrs | | | | | | | | |

### Example 14

Criticality of Oleyl Alcohol level. PEG-based Ointment formulations containing 0%, 1′)/0 and 2% oleyl alcohol were tested for in vitro percutaneous skin absorption. Based on in vitro percutaneous absorption testing, the amount of tofacitinib permeated over time increases according to the level of oleyl alcohol in the formulation. The ointment composition without oleyl alcohol is same as Ointment 2 in Table 28. The ointment composition with 2% oleyl alcohol is the same as Ointment 1 in Table 28.

TABLE 33
Tofacitinib Flux through Human Cadaver Skin with PEG-based ointment
formulations containing various levels of oleyl alcohol

| | | PEG based | PEG based |
|---|---|---|---|
| | PEG based | Ointment | Ointment |
| | Ointment | with oleyl | with oleyl |
| | without oleyl | alcohol | alcohol |
| Skin Flux Result Summary | alcohol | w/1% OA | w/2% OA |
| Number of cells | 6 | 5 | 5 |
| Penetrated in 48 hrs, ug/cm2 | 1.58 | 1.9 | 4.6 |
| % RSD (@ 48 hrs) | 40.3 | 88.2 | 93.9 |
| % of applied dose (@ 48 hrs) | 1% | 1.5% | 4.3% |

### Example 15

The present inventors found that tofacitinib has poor stability in the presence of polyethylene glycol (PEG). It was surprisingly discovered that the tofacitinib stability can be improved if glycerin was added to the formulation. The following data demonstrate this enhanced stability.

TABLE 34
Potency result of Tofacitinib in PEG-based ointment
formulations after storage at 40° C.

| | Tofacitinib Potency (% of theoretical) | |
|---|---|---|
| | PEG-based ointment | PEG-based ointment with |
| | without Glycerin | 20% Glycerin |
| Initial | 100.0 | 99.9 |
| 2 wks @ 40° C. | 95.4 | 97.4 |
| 4 wks @ 40° C. | 91.2 | 97.3 |

Addition of antioxidants further improved tofacitinib stability in the presence of polyethylene glycol (PEG). In a study whereby binary mixtures of tofacitinib and PEG 400 or PEG 3350 were prepared and evaluated for stability after storage at 60° C. Table 35 present date showing that addition of antioxidants result further improved tofacitinib stability.

TABLE 35
Potency result of Tofacitinib in binary mixture
of Tofacitinib and PEG after storage at 60° C.

| | | Time Point | Tofacitinib |
|---|---|---|---|
| | | (Days at | Potency |
| Binary Mixture | Sample | 60° C.) | (% of theory) |
| PEG 400 and | Without antioxidant | 4 | 84.4 |
| tofacitinib | | 7 | 71.0 |
| | With 0.1% BHA | 4 | 90.3 |
| | | 7 | 80.8 |
| | With 0.1% BHA and | 4 | 95.1 |
| | 0.05% Propyl Gallate | 7 | 93.6 |
| PEG 3350 and | Without antioxidant | 4 | 59.5 |
| tofacitinib | | 7 | 50.1 |
| | With 0.1% BHA | 4 | 94.8 |
| | | 7 | 93.8 |

### In-vitro Percutaneous Flux Method:

The Hanson Microette automated diffusion cell system was used to generate data for the in-vitro percutaneous flux experiments. Small sections of human cadaver skin were mounted on the diffusion cells and equilibrated to reach a skin surface temperature of 32° C. The partial-media replacement procedure was employed and it consisted of aliquot sampling of the receptor cell contents, followed by equal volume replacement of the sampled media. Samples were collected at 2, 4, 8, 12, 20 24, 30, 36, and 48 hrs to generate cumulative permeation and flux profiles. Phosphate buffered saline with 0.1% gentamicin preservative was used as the receptor media. A finite dose of approximately 10 mg of ointment sample was applied to cover the entire surface of the skin. The receptor media samples were assayed using a suitable HPLC method for tofacitinib content.

What is claimed is:

1. A crystalline form of 3-((3R,4R)-4-methyl-3-[methyl-(7H-pyrrolo[2,3-d]pyrimidin-4-yl)-amino]-piperidin-1-yl)-3-oxopropionitrile, having a powder X-ray diffraction pattern comprising peaks, in terms of 2θ, at 6.4°, 14.3°, and 17.0° 2θ±0.2° 2θ.

2. The crystalline form of claim 1, having solid state ¹³C nuclear magnetic resonance chemical shifts selected from the group consisting of: 63.1; 63.1 and 68.6; 68.6; 18.8 and 60.1; 18.8; 60.1; 63.1 and 162.1 and 162.1 ppm±0.2 ppm.

3. The crystalline form of claim 1, wherein the 2-propanol level is from 2.6 to 2.9% weight.

4. The crystalline form of claim 1, wherein the water level is from 0.5 to 4.0% weight.

5. A crystalline form of 3-((3R,4R)-4-methyl-3-[methyl-(7H-pyrrolo[2,3-d]pyrimidin-4-yl)-amino]-piperidin-1-yl)-3-oxopropionitrile, having a powder X-ray diffraction pattern comprising peaks, in terms of 2θ, at 6.4°, 9.1°, and 11.1° 2θ±0.2° 2θ.

6. The crystalline form of claim 5, having solid state ¹³C nuclear magnetic resonance chemical shifts selected from the group consisting of: 63.1; 63.1 and 68.6; 68.6; 18.8 and 60.1; 18.8; 60.1; 63.1 and 162.1 and 162.1 ppm±0.2 ppm.

7. The crystalline form of claim 5, wherein the 2-propanol level is from 2.6 to 2.9% weight.

8. The crystalline form of claim 5, wherein the water level is from 0.5 to 4.0% weight.

9. A crystalline form of 3-((3R,4R)-4-methyl-3-[methyl-(7H-pyrrolo[2,3-d]pyrimidin-4-yl)-amino]-piperidin-1-yl)-3-oxopropionitrile, having solid state ¹³C nuclear magnetic resonance chemical shifts at 157.0, 151.0, 102.4, 44.8 and 32.7 ppm±0.2 ppm.

10. The crystalline form of claim 9, wherein the 2-propanol level is from 2.6 to 2.9% weight.

11. The crystalline form of claim 9, wherein the water level is from 0.5 to 4.0 weight.

12. A non-crystalline form of 3-((3R,4R)-4-methyl-3-[methyl-(7H-pyrrolo[2,3-d]pyrimidin-4-yl)-amino]-piperidin-1-yl)-3-oxopropionitrile character-ized by a physical or spectroscopic analysis selected from the group consisting of:
a) a solid state ¹³C nuclear magnetic resonance spectrum comprising chemical shifts at 161.9, 152.0, 103.3, 31.8, and 26.0 ppm±0.2 ppm.;
b) a set of Raman bands at 1311, 1506, and 2258 cm⁻¹±2 cm⁻¹; and,
c) a set of infrared bands at 1407, 1554, and 1647 cm⁻¹±2 cm⁻¹.

13. A pharmaceutical composition comprising: 3-((3R,4R)-4-methyl-3-[methyl-(7H-pyrrolo[2,3-d]pyrimidin-4-yl)-amino]-piperidin-1-yl)-3-oxopropionitrile; one or more penetration enhancer; and, a pharmaceutically acceptable carrier.

14. The pharmaceutical composition of claim 13 wherein 3-((3R,4R)-4-methyl-3-[methyl-(7H-pyrrolo[2,3-d]pyrimidin-4-yl)-amino]-piperidin-1-yl)-3-oxo-propionitrile has a crystalline form or a non-crystalline form.

15. The pharmaceutical composition of claim 13, comprising a topical formulation selected from a cream, transdermal patch, ointment, ophthalmic drops, lotion and gel.

16. The pharmaceutical composition of any of claims 13 wherein the topical formulation contains about 0.1%-5.0% (w/v) 3-((3R,4R)-4-methyl-3-[methyl-(7H-pyrrolo[2,3-d]pyrimidin-4-yl)-amino]-piperidin-1-yl)-3-oxopropionitrile.

17. The pharmaceutical composition of claim 15 wherein the topical formulation contains about 0.5%-2.3% (w/v) 3-((3R,4R)-4-methyl-3-[methyl-(7H-pyrrolo[2,3-d]pyrimidin-4-yl)-amino]-piperidin-1-yl)-3-oxopropionitrile.

18. The pharmaceutical composition of claim 15 wherein the topical formulation contains about 2.0% (w/v) 3-((3R,4R)-4-methyl-3-[methyl-(7H-pyrrolo[2,3-d]pyrimidin-4-yl)-amino]-piperidin-1-yl)-3-oxopropionitrile.

19. The pharmaceutical composition of claim 13, wherein said penetration enhancer is selected from saturated C10-C18 fatty alcohols, cis-unsaturated C10-C18 fatty alcohols, C10-C18 saturated fatty acids; and, C10-C18 cis-unsaturated fatty acid.

20. The pharmaceutical composition of claim 13, wherein the pharmaceutically acceptable carrier is at least 30% by weight PEG, and further comprising stabilizing excipients in an amount sufficient to achieve a chemically stable formulation such that the level of total degradants is not more that 7% by weight after 4 weeks at 40° C.

21. The pharmaceutical composition of claim 13, wherein the pharmaceutically acceptable carrier is at least 30% by weight PEG, and further comprising one or more aldehyde scavenger or anti-oxidant excipient in an amount sufficient to achieve a chemically stable formulation such that the level of total degradants is not more that 7% by weight after 4 weeks at 40° C.

22. The pharmaceutical composition of claim 13, further comprising an aldehyde scavenger selected from glycerin and propylene glycol and an anti-oxidant selected from butylated hydroxyanisole, butylated hydroxytoluene, propyl gallate, ascorbic acid, polyphenols, tocopherols, and their derivatives.

23. The pharmaceutical composition of claim 13, wherein said penetration enhancer is selected from oleyl alcohol, linoleyl alcohol, γ-linolenyl alcohol, linolenyl alcohol, decyl alcohol, lauryl alcohol, myristyl alcohol, cetyl alcohol, stearyl alcohol, capric acid, lauric acid, myristic acid, palmitic acid, stearic acid and arachidic acid, palmitoleic acid, oleic acid, cis-vaccenic acid, linoleic acid, γ-linolenic acid, linolenic acid, and arachidonic acid.

24. The pharmaceutical composition of claim 13, wherein said penetration enhancer is oleyl alcohol.

25. The pharmaceutical composition of claim 13, wherein characterized by a percutaneous flux that is equal or greater than the flux measured from a composition consisting by weight of about 2% tofacitinib free base, about 1.8% oleyl alcohol, about 17.9% glycerine, about 18% propylene glycol, about 30% PEG 400, about 30% PEG 3350, and about 0.1% BHA.

26. A pharmaceutical composition comprising: about 2.0% 3-((3R,4R)-4-methyl-3-[methyl-(7H-pyrrolo[2,3-d]pyrimidin-4-yl)-amino]-piperidin-1-yl)-3-oxopropionitrile by weight, about 2.0% oleyl alcohol, about 20.0% glycerin, at least about 30.0% polyethylene glycol, and about 0.1% butyl hydroxyanisole

27. A method of topically treating a disease in a mammal, comprising administering by a topical mode of administration to a mammal in need thereof a therapeutically effective amount of 3-((3R,4R)-4-methyl-3-[methyl-(7H-pyrrolo[2,3-d]pyrimidin-4-yl)-amino]-piperidin-1-yl)-3-oxopropionitrile, having a crystalline form or non-crystalline form, or a pharmaceutically acceptable salt thereof, one or more penetration enhancer; and a pharmaceutically acceptable carrier, wherein the disease is selected from the group consisting of psoriasis and dermatitis and said penetration enhancer is selected from oleyl alcohol, linoleyl alcohol, γ-linolenyl alcohol, linolenyl alcohol, decyl alcohol, lauryl alcohol, myristyl alcohol, cetyl alcohol, stearyl alcohol, capric acid, lauric acid, myristic acid, palmitic acid, stearic acid and arachidic acid, palmitoleic acid, oleic acid, cis-vaccenic acid, linoleic acid, γ-linolenic acid, linolenic acid, and arachidonic acid.

28. The method of claim 27, wherein the disease is psoriasis, and the penetration enhancer is oleyl alcohol.
